(19) United States
(12) Patent Application Publication
(10) Pub. No.: US 2023/0240589 A1
(43) Pub. Date: Aug. 3, 2023

(54) SENSING SYSTEMS AND METHODS FOR DIAGNOSING, STAGING, TREATING, AND ASSESSING RISKS OF LIVER DISEASE USING MONITORED ANALYTE DATA

(71) Applicant: Dexcom, Inc., San Diego, CA (US)

(72) Inventors: Partha Pratim RAY, San Diego, CA (US); Matthew L. JOHNSON, San Diego, CA (US); Qi AN, San Diego, CA (US); Jason M. HALAC, San Diego, CA (US); Rush BARTLETT, Austin, TX (US); John PADERI, San Francisco, CA (US)

(21) Appl. No.: 18/163,829

(22) Filed: Feb. 2, 2023

Related U.S. Application Data
(60) Provisional application No. 63/267,447, filed on Feb. 2, 2022.
(60) Provisional application No. 63/403,568, filed on Sep. 2, 2022.
(60) Provisional application No. 63/403,582, filed on Sep. 2, 2022.

(51) Int. Cl. A61B 5/00; A61B 5/145; A61B 5/1486; G16H 10/60; G16H 40/67

(52) U.S. Cl. CPC A61B 5/4244; A61B 5/14546; A61B 5/14532; A61B 5/14865; A61B 5/7275; A61B 5/7246; A61B 5/7289; A61B 5/4866; A61B 5/7267; A61B 5/4848; G16H 10/60; G16H 40/67

(57) ABSTRACT

Certain aspects of the present disclosure relate to methods and systems for generating and utilizing analyte measurements. In certain aspects, a monitoring system comprises a continuous analyte sensor configured generate analyte measurements associated with analyte levels of a patient and a sensor electronics module coupled to the continuous analyte sensor and configured to receive and process the analyte measurements.

### CROSS-REFERENCE TO RELATED APPLICATIONS

This application claims priority to and benefit of U.S. Provisional Application No. 63/267,447, filed Feb. 2, 2022, U.S. Provisional Application No. 63/403,568, filed Sep. 2, 2022, and U.S. Provisional Application No. 63/403,582, filed Sep. 2, 2022, which are hereby assigned to the assignee hereof and hereby expressly incorporated by reference in their entirety as if fully set forth below and for all applicable purposes.

### BACKGROUND

Liver disease, also referred to as hepatic disease, is any disturbance of liver function that causes illness. The liver is responsible for many critical functions within the human body from protein production and blood clotting to cholesterol, lactate, glucose, and iron metabolism. Should the liver become diseased or injured, the impairment or loss of these functions can cause significant damage to the human body.

Liver disease is generally classified as either acute or chronic based upon the duration of the disease. Liver disease may be caused by infection, injury, exposure to drugs or toxic compounds, alcohol, impurities in foods, abnormal build-up of normal substances in the blood stream, an autoimmune process, a genetic defect (e.g., such as haemochromatosis), and/or or unknown cause(s). Common liver diseases include cirrhosis, liver fibrosis, non-alcoholic fatty liver disease (NAFLD), non-alcoholic steatohepatitis (NASH), hepatic ischemia reperfusion injury, primary biliary cirrhosis (PBC), and hepatitis.

The American Liver Foundation estimates that more than 20% of the population has NAFLD. It is suggested that obesity, unhealthy diets, and sedentary lifestyles may contribute to the high prevalence of NAFLD. When left untreated, NAFLD may progress to NASH, causing serious adverse effects to the body. Once NASH is developed, a person may experience liver swelling and scarring (i.e., cirrhosis) over time.

Lactate is measured and analyzed using various approaches including central laboratory methods, near patient blood gas analysis, and analysis using portable point of care (POC) handheld devices. The central laboratory approach involves transportation of blood samples of a patient to a laboratory via porters or air-tube systems. Unfortunately, the central laboratory approach often suffers from prolonged times between when blood is drawn to when the clinician becomes aware of the test results, resulting in a potential delay in clinical decision-making.

As POC technology has advanced, near-patient, benchtop blood gas analyzers have been made available for lactate testing. However these devices are not portable and their availability is usually restricted to individual specialized units, e.g., emergency departments (EDs) and intensive care units (ICUs). Further, sample turnaround time of test results may be affected by delays in transportation to the ED or ICU, when the sample was drawn outside these major units.

For this reason, small hand-held devices, much like glucose meters, have been made available for lactate measuring and analysis. A user may carry a self-monitoring lactate monitor which typically requires the user to prick his or her finger to measure his or her lactate levels. However, given the inconvenience associated with traditional finger pricking methods, it is unlikely that a user will take a timely lactate measurement. Consequently, the user's lack of engagement with the device can have devastating results. In particular, a user who forgoes engaging with the device may also fail to manage their condition outside of the device's use. Where the condition is left unmanaged for too long, the user's liver condition may significantly deteriorate, additional health issues may arise, and, in some cases, lead to an increased risk or likelihood of mortality.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example decision support system that may be used in connection with implementing embodiments of the present disclosure.

FIG. 2 is a diagram conceptually illustrating an example continuous analyte monitoring system including example continuous analyte sensor(s) with sensor electronics, in accordance with certain aspects of the present disclosure.

FIG. 3 illustrates example inputs and example metrics that are calculated based on the inputs for use by the decision support system of FIG. 1, according to some embodiments disclosed herein.

FIG. 4 is a flow diagram illustrating an example method for providing decision support using a continuous analyte sensor including, at least, a continuous lactate sensor, in accordance with some example aspects of the present disclosure.

FIG. 5 is an example workflow for determining a liver lactate clearance rate using at least, a continuous lactate monitor, according to certain embodiments of the present disclosure.

FIG. 6 is a flow diagram depicting a method for training machine learning models to provide a prediction of liver disease diagnosis, according to certain embodiments of the present disclosure.

FIG. 7 is a block diagram depicting an example computing device configured to execute a decision support engine, according to certain embodiments of the present disclosure.

FIGS. 8A-8B depict exemplary enzyme domain configurations for a continuous multi-analyte sensor, according to certain embodiments of the present disclosure.

FIGS. 8C-8D depict exemplary enzyme domain configurations for a continuous multi-analyte sensor, according to certain embodiments of the present disclosure.

FIG. 8E depicts an exemplary enzyme domain configuration for a continuous multi-analyte sensor, according to certain embodiments of the present disclosure.

FIGS. 9A-9B depict alternative views of an exemplary dual electrode enzyme domain configuration for a continuous multi-analyte sensor, according to certain embodiments of the present disclosure.

FIGS. 9C-9D depict alternative views of an exemplary dual electrode enzyme domain configuration for a continuous multi-analyte sensor, according to certain embodiments of the present disclosure.

FIG. 9E depicts an exemplary dual electrode configuration for a continuous multi-analyte sensor, according to certain embodiments of the present disclosure.

FIG. 10A depicts an exemplary enzyme domain configuration for a continuous multi-analyte sensor, according to certain embodiments of the present disclosure.

FIGS. 10B-10C depict alternative exemplary enzyme domain configurations for a continuous multi-analyte sensor, according to certain embodiments of the present disclosure.

FIG. 11 depicts an exemplary enzyme domain configuration for a continuous multi-analyte sensor, according to certain embodiments of the present disclosure.

FIGS. 12A-12D depict alternative views of exemplary dual electrode enzyme domain configurations G1-G4 for a continuous multi-analyte sensor, according to certain embodiments of the present disclosure.

FIG. 13A depicts an exemplary lactate sensor, according to certain embodiments of the present disclosure.

FIG. 13B depicts a cross-sectional view of the electroactive section of the example lactate sensor of FIG. 13A, according to certain embodiments of the present disclosure.

FIGS. 14A-14C depict an exemplary embodiment of a continuous analyte sensor system implemented as a wearable lactate sensor, according to certain embodiments of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Liver disease is not easily discoverable. In particular, the liver is sometimes referred to as a silent organ as, even when liver failure occurs, the symptoms often go unnoticed. In some cases, when symptoms, such as jaundice, for example, become apparent, the liver disease may have already reached an advanced stage. Accordingly, early liver disease diagnosis and staging is vital to effectively treat, and in some cases, reverse the disease.

Disease diagnosis is the act or process of identifying or determining the nature and cause of a disease, while disease staging is a clinically based measure of severity that uses objective medical criteria to assess the stage of the identified disease's progression. More specifically, disease staging may provide important information about the extent of liver disease in a patient's body and an anticipated response of a patient to different forms of treatment.

Physicians use information from a patient's history, physical examination, laboratory findings, and other diagnostic tests to diagnose and stage a disease to prescribe appropriate treatment. For example, liver function tests may be used by physicians to screen for liver infection, monitor the progression of liver disease, assess the effectiveness of different treatments for liver disease, and monitor the possible side effects of medication to a patient's liver, to name a few. Liver function tests check the levels of certain enzymes and proteins in a patient's blood. Levels that are higher or lower than normal can, in some cases, indicate liver problems.

However, such conventional liver disease diagnostic and staging methods face many challenges with respect to efficiency, accuracy, and delay in providing liver diagnosis for treatment decision-making. For example, it can be very difficult to determine which particular diagnosis is indicated by a particular combination of symptoms, especially if symptoms are nonspecific, such as fatigue. Liver disease may also present atypically, with an unusual and unexpected constellation of symptoms. Currently, the standard of care for definitive diagnosis of liver disease is a liver biopsy. A liver biopsy is a non-scalable and invasive way to diagnose liver disease. Accordingly, making an accurate diagnosis can prove to be particularly challenging for physicians. Further, when a patient seeks health care, there is an iterative process of information gathering, information integration and interpretation, and determining a working diagnosis, and throughout the diagnostic process, there is an ongoing assessment of whether sufficient information has been collected. If a physician is not satisfied that the necessary information has been collected to explain the patient's health problem or accurately diagnose the patient with liver disease, or that the information available is not consistent with a liver disease diagnosis, then the process of information gathering, information integration and interpretation, and developing a working diagnosis continues. Accordingly, diagnosis of the liver disease may be delayed, and in some cases, contribute to a patient experiencing worsening symptoms, a decline in overall health, and even death given the time-dependent nature of many diseases, including liver disease.

Further, existing technologies, such as point of contact (POC) devices, have been introduced to enable timely assessment of patients with, or at risk, of liver disease. As mentioned previously, one such POC device may include a portable, self-monitoring lactate monitor which typically requires the user to prick his or her finger to give a single standalone reading indicative of his or her lactate levels for diagnosing liver health. Thus far, POC devices have almost exclusively included diagnostic devices—devices that can analyze a patient to give a single standalone reading. As such, existing devices suffer from a technical problem of failing to continuously (and/or semi-continuously and/or periodically) monitor the concentration of changing analytes, such as lactate, to give a continuous (and/or semi-continuous and/or periodic) readout. Such continuous monitoring of analytes is advantageous in diagnosing and staging a disease of a patient given the continuous measurements provide continuously up to date measurements as well as information on the trend and rate of analyte change over a continuous period.

Continuous measurements as proposed herein, provide a more accurate indication of liver metabolic function and liver health as compared to a single point in time reading. A single point in time reading may be influenced by a patient's activity, such as exercise or diet changes near or during the point in time. Additionally, imaging techniques that determine structural aspects of the liver do not provide information on the metabolic functional performance of liver cellular tissue. Measuring analytes (e.g., lactate) in a continuous readout as proposed herein may increase understanding of metabolic function of the liver to confirm good liver performance, or determine the presence and/or magnitude of liver metabolic dysfunction. Such information may also be used to make more informed decisions in the assessment of liver health and treatment of liver disease. As a result of this technical problem, diagnosing liver disease, or a risk thereof, may not be accurate, which, in some cases, might prove to be life threatening for a patient with liver disease.

Accordingly, certain embodiments described herein provide a technical solution to the technical problem described above by providing decision support around liver disease using a continuous analyte monitoring system, including, at least, a continuous lactate sensor. As used herein, the term “continuous” may mean fully continuous, semi-continuous, periodic, etc. The decision support may be provided in the form of risk assessment, diagnosis, staging, and/or recommendations for treatment of liver disease, as described in more detail herein. As used herein, risk assessment may refer to the evaluation or estimation of liver disease of a patient reaching a more advanced stage, mortality risk, liver cancer risk, and the like.

In certain embodiments, the continuous analyte monitoring system may provide decision support to a patient based on a variety of collected data, including analyte data, patient information, secondary sensor data (e.g., non-analyte data), etc. For example, the analyte data may include continuously monitored lactate data in addition to other continuously monitored analyte data, such as glucose, ketones, and potassium.

The continuously monitored lactate data may indicate, or be used for determining, the patient's lactate levels, lactate production rates, lactate metabolism, and/or lactate clearance rates. Certain embodiments of the present disclosure provide techniques and systems for more accurately determining a patient's lactate clearance rate using the continuously monitored lactate data as well as correcting a patient's lactate clearance rate by using measurements associated with the non-analyte sensor data and/or other patient information, as further described below. As described above, the collected data also includes patient information, which may include information related to age, gender, family history of liver disease, other health conditions, etc. Secondary sensor data may include accelerometer data, heart rate data, temperature, blood pressure, or any other sensor data other than analyte data.

According to embodiments of the present disclosure, the decision support system presented herein is designed to provide a diagnosis for patients with, or at risk of, liver disease as well as disease decision support to assist the patient in managing their liver disease, or a risk thereof. Providing liver disease decision support may involve using large amounts of collected data, including for example, the analyte data, patient information, and secondary sensor data mentioned above, to (1) automatically detect and classify abnormal liver conditions, (2) assess the presence and severity of liver disease, (3) risk stratify patients to identify those patients with a high risk of liver disease, (4) identify risks (e.g., mortality risk, liver cancer risk, etc.) associated with a current liver disease diagnosis, (5) make patient-specific treatment decisions or recommendations for liver disease, and (6) provide information on the effect of an intervention (e.g., an effect of a lifestyle change of the patient, an effect of a surgical procedure, an effect of the patient taking new medication, etc.). In other words, the decision support system presented herein may offer information to direct and help improve care for patients with, or at risk, of liver disease.

In certain embodiments, the decision support system described herein may use various algorithms or artificial intelligence (AI) models, such as machine-learning models, trained based on patient-specific data and/or population data to provide real-time decision support to a patient based on the collected information about the patient. For example, certain aspects are directed to algorithms and/or machine-learning models designed to assess the presence and severity of liver disease in a patient. The algorithms and/or machine-learning models may be used in combination with one or more continuous analyte sensors, including at least a continuous lactate sensor, to provide liver disease assessment and staging, for example, at a regular intervals (e.g., daily, weekly, etc.). In particular, the algorithms and/or machine-learning models may take into account parameters, such as lactate clearance rate (including lactate half-life), lactate levels, lactate rate of change, fasting lactate, postprandial lactate, lactate production rates, and lactate baselines of a patient, when diagnosing and staging liver disease.

Based on these parameters, the algorithms and/or machine-learning models may provide a risk assessment of one or more liver disease types and severity, as well the progression a patient has made towards one or more of those liver disease types. The algorithms and/or machine-learning models may take into consideration population data, personalized patient-specific data, or a combination of both when diagnosing and staging liver disease for a patient.

According to certain embodiments, prior to deployment, the machine learning models are trained with training data, e.g., including population data. As described in more detail herein, the population data may be provided in a form of a dataset including data records of historical patients with varying stages of liver disease. Each data record is then featurized (e.g., refined into a set of one or more features, or predictor variables) and labeled. Data labeling is the process of adding one or more meaningful and informative labels to provide context to the data for learning by the machine learning models. In certain embodiments, each data record is labeled with its corresponding liver disease diagnosis, assigned disease score, risk assessment, etc. The features associated with each data record may be used as input into the machine learning models, and the generated output may be compared to label(s) assigned to each of the data records. The models may compute a loss based on the difference between the generated output and the provided label. This loss can then be used to modify the internal parameters or weights of the models. By iteratively processing features associated with each data record corresponding to each historical patient, the models may be iteratively refined to generate accurate predictions of liver disease presence and severity in a patient.

The combination of a continuous analyte monitoring system with machine learning models and/or algorithms for diagnosing, staging, and assessing risk of liver disease provided by the decision support system described herein enables real-time diagnosis to allow early intervention. In particular, the decision support system may be used to provide an early alert of liver decompensation and/or deliver information about other complications related to the liver. Early detection of such decompensation and/or other complications may allow for intervention at the earliest possible stage to ultimately improve liver disease outcomes. For example, early intervention may reduce hospitalization, complications, and death, in some cases. In addition, baseline lactate levels and changes in lactate levels provided by the continuous analyte monitoring system may be used as input into the machine learning models and/or algorithms to triage patients for more urgent care. In patients at risk for sepsis or septic shock, traumatic brain injury, acute kidney injury, hepatic encephalopathy, or end stage liver disease, increases in lactate may be used to inform urgent medical intervention.

In addition, through the combination of a continuous analyte monitoring system with machine learnings and/or algorithms for diagnosing, staging, and assessing risk of liver disease, the decision support system described herein may provide the necessary accuracy and reliability patients expect. For example, biases, human errors, and emotional influence may be minimized when assessing the presence and severity of liver disease in patients. Further, machine learning models and algorithms in combination with analyte monitoring systems may provide insight into patterns and or trends of decreasing health of a patient, at least with respect to the liver, which may have been previously missed. Accordingly, the decision support system described herein may assist in the identification of liver health for diagnosis, preventive, and treatment purposes.

### Example Decision Support System Including an Example Analyte Sensor for Diagnosing, Staging, Treating, and Assessing Risks of Liver Disease

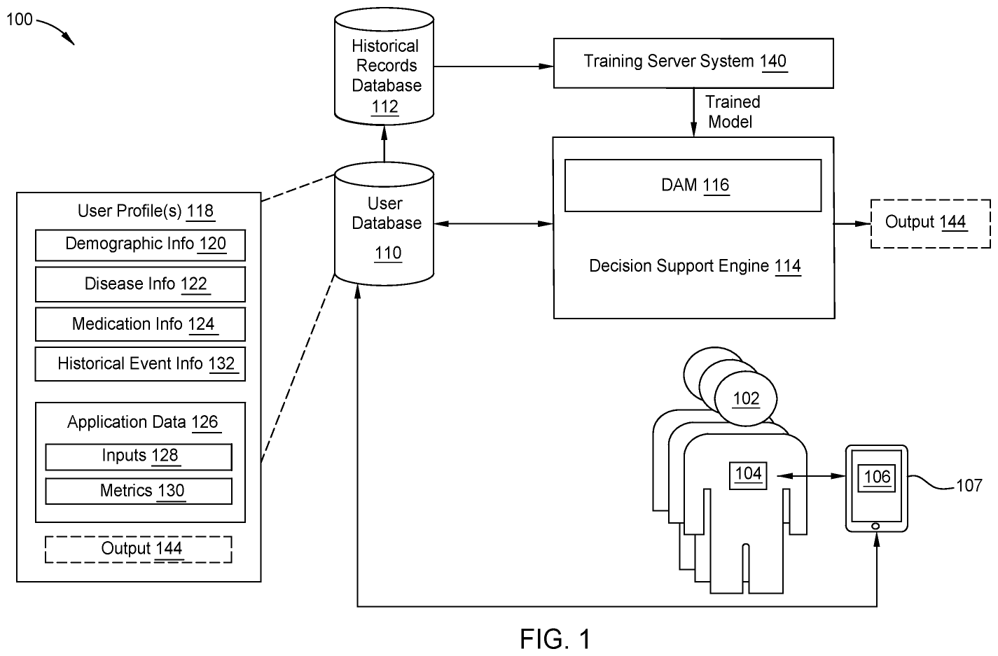

FIG. 1 illustrates an example decision support system (also referred to as a “monitoring system”) 100 for diagnosing, staging, treating, and assessing risks of liver disease of users 102 (individually referred to herein as a user and collectively referred to herein as users), using a continuous analyte monitoring system 104, including, at least, a continuous lactate sensor. A user, in certain embodiments, may be the patient or, in some cases, the patient's caregiver. In certain embodiments, system 100 includes continuous analyte monitoring system 104, a display device 107 that executes application 106, a decision support engine 114, a user database 110, a historical records database 112, a training server system 140, and a decision support engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods may include, but may not be limited to, potassium, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-peptide; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, a challenge agent analyte (e.g., introduced for the purpose of measuring the increase and or decrease in rate of change in concentration of the challenge agent analyte or other analytes in response to the introduced challenge agent analyte), or a drug or pharmaceutical composition, including but not limited to insulin (e.g., exogenous or endogenous); glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include lactate, and in some cases glucose, ketone and/or potassium, other analytes listed, but not limited to, above may also be considered.

In certain embodiments, continuous analyte monitoring system 104 is configured to continuously measure one or more analytes and transmit the analyte measurements to an electric medical records (EMR) system and/or an interface engine (not shown in FIG. 1). An EMR system is a software platform which allows for the electronic entry, storage, and maintenance of digital medical data. An interface engine is a data synchronization tool to ensure that EMR databases and other systems databases are in sync on a network. An EMR system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system may also be used to create reports for clinical care and/or disease management for a patient.

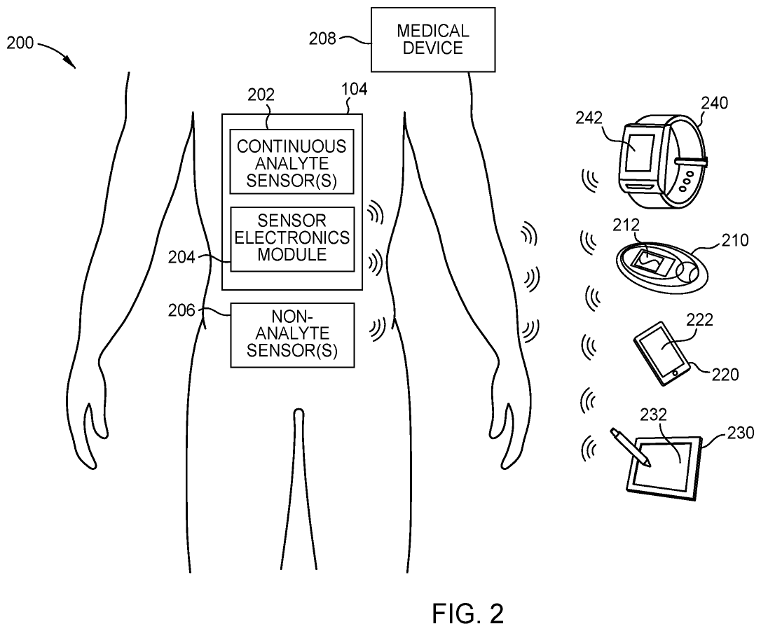

In certain embodiments, continuous analyte monitoring system 104 is configured to continuously measure one or more analytes and transmit the analyte measurements to display device 107 for use by application 106. In some embodiments, continuous analyte monitoring system 104 transmits the analyte measurements to display device 107 through a wireless connection (e.g., Bluetooth connection, WiFi connection and/or NFC). The transmission of analyte measurements may be broadcast or on-demand and continuous (e.g., fully continuous, semi-continuous, or periodic). In certain embodiments, display device 107 is a smart phone. However, in certain other embodiments, display device 107 may instead be any other type of computing device such as a laptop computer, a smart watch, a tablet, or any other computing device capable of executing application 106. Continuous analyte monitoring system 104 may be described in more detail with respect to FIG. 2.

Application 106 is a mobile health application that is configured to receive and analyze analyte measurements from analyte monitoring system 104. In particular, application 106 stores information about a user, including the user's analyte measurements, in a user profile 118 associated with the user for processing and analysis, as well as for use by decision support engine 114 to provide decision support recommendations or guidance to the user.

Decision support engine 114 refers to a set of software instructions with one or more software modules, including data analysis module (DAM) 116. In certain embodiments, decision support engine 114 executes entirely on one or more computing devices in a private or a public cloud. In such embodiments, application 106 communicates with decision support engine 114 over a network (e.g., Internet). In some other embodiments, decision support engine 114 executes partially on one or more local devices, such as display device 107, and partially on one or more computing devices in a private or a public cloud. In some other embodiments, decision support engine 114 executes entirely on one or more local devices, such as display device 107 or analyte sensor system 104 (e.g., sensor electronics module 204 of FIG. 2). As discussed in more detail herein, decision support engine 114 may provide decision support recommendations to the user via application 106. Decision support engine 114 provides decision support recommendations based on information included in user profile 118.

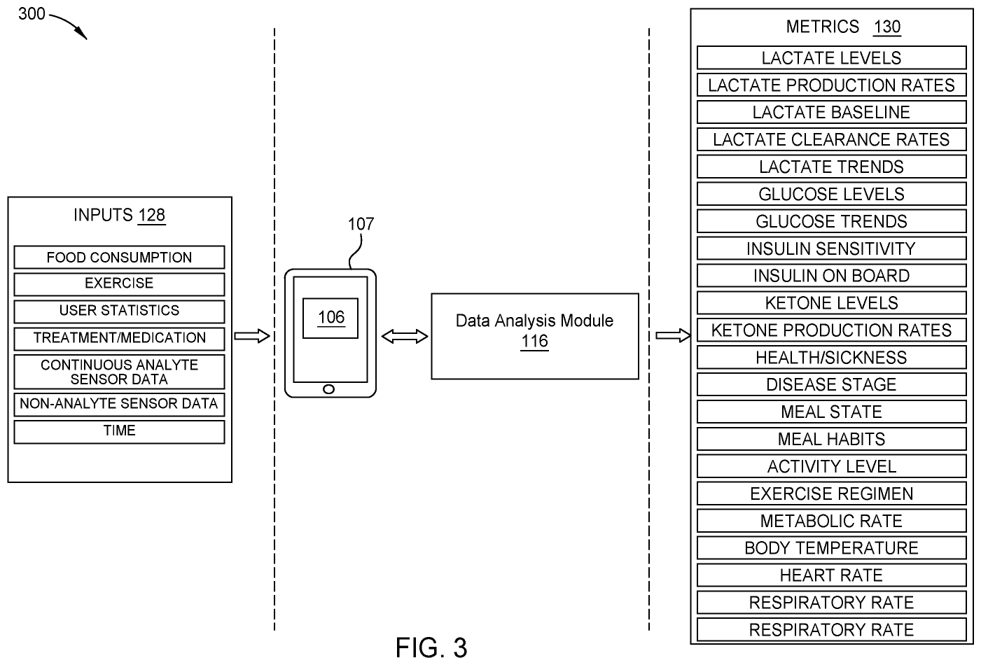

User profile 118 may include information collected about the user from application 106. For example, application 106 provides a set of inputs 128, including the analyte measurements received from continuous analyte monitoring system 104, that are stored in user profile 118. In certain embodiments, inputs 128 provided by application 106 include other data in addition to analyte measurements received from continuous analyte monitoring system 104. For example, application 106 may obtain additional inputs 128 through manual user input, one or more other non-analyte sensors or devices, other applications executing on display device 107, etc. Non-analyte sensors and devices include one or more of, but are not limited to, an insulin pump, respiratory sensor, sensors or devices provided by display device 107 (e.g., accelerometer, camera, global positioning system (GPS), heart rate monitor, etc.) or other user accessories (e.g., a smart watch), or any other sensors or devices that provide relevant information about the user. Inputs 128 of user profile 118 provided by application 106 are described in further detail below with respect to FIG. 3.

DAM 113 of decision support engine 114 is configured to process the set of inputs 128 to determine one or more metrics 130. Metrics 130, discussed in more detail below with respect to FIG. 3, may, at least in some cases, be generally indicative of the health or state of a user, such as one or more of the user's physiological state, trends associated with the health or state of a user, etc. In certain embodiments, metrics 130 may then be used by decision support engine 114 as input for providing guidance to a user. As shown, metrics 130 are also stored in user profile 118.

User profile 118 also includes demographic info 120, disease progression info 122, and/or medication info 124. In certain embodiments, such information may be provided through user input or obtained from certain data stores (e.g., electronic medical records (EMRs), etc.). In certain embodiments, demographic info 120 may include one or more of the user's age, body mass index (BMI), ethnicity, gender, etc. In certain embodiments, disease progression info 122 may include information about a disease of a user, such as whether the user has been previously diagnosed with cirrhosis, liver fibrosis, NAFLD, NASH, hepatic ischemia reperfusion injury, primary biliary cholangitis (PBC), primary sclerosing cholangitis (PSC), or whether the user has been previously diagnosed with liver disease caused by viruses, such as hepatitis A, hepatitis B, or hepatitis C. In certain embodiments, information about a user's disease may also include the length of time since diagnosis, the level of disease control, level of compliance with liver disease management therapy, predicted liver function, other types of diagnosis (e.g., heart disease, obesity) or measures of health (e.g., heart rate, exercise, stress, sleep, etc.), and/or the like.

In certain embodiments, medication info 124 may include information about the amount, frequency, and type of a medication taken by a user.

In certain embodiments, medication information may include information about the consumption of one or more drugs known to damage the liver (e.g., affect lactic clearance) and/or lead to liver toxicity. One or more drugs known to damage the liver and/or lead to liver toxicity may include antibiotics such as amoxicillin/clavulanate, clindamycin, erythromycin, nitrofurantoin, rifampin, sulfonamides, tetracyclines, trimethoprim/sulfamethoxazole, and drugs used to treat tuberculosis (isoniazid and pyrazinamide), anticonvulsants such as tarbamazepine, thenobarbital, phenytoin, and valproate, antidepressants such as bupropion, fluoxetine, mirtazapine, paroxetine, sertraline, trazodone, and tricyclic antidepressants such as amitriptyline, antifungal drugs such as ketoconazole and terbinafine, antihypertensive drugs (e.g., drugs used to treat high blood pressure or sometimes kidney or heart disorder) such as captopril, enalapril, irbesartan, lisinopril, losartan, and verapamil, antipsychotic drugs such as phenothiazines (e.g., such as chlorpromazine) and risperidone, heart drugs such as amiodarone and clopidogrel, hormone regulation drugs such as anabolic steroids, birth control pills (oral contraceptives), and estrogens, pain relievers such as acetaminophen and nonsteroidal anti-inflammatory drugs (NSAIDs), and other drugs such as acarbose (e.g., used to treat diabetes), allopurinol (e.g., used to treat gout), antiretroviral therapy (ART) drugs (e.g., used to treat human immunodeficiency virus (HIV) infection), baclofen (e.g., a muscle relaxant), cyproheptadine (e.g., an antihistamine), azathioprine (e.g., used to prevent rejection of an organ transplant), methotrexate (e.g., used to treat cancer), omeprazole (e.g., used to treat gastroesophageal reflux), PD-1/PD-L1 inhibitors (e.g., anticancer drugs), statins (e.g., used to treat high cholesterol levels), and many types of chemotherapy, including immune checkpoint inhibitors.

In certain embodiments, medication information may include information about consumption of one or more drugs known to improve liver function. One or more drugs known to improve liver function may include ademetionine, avatrombopag, dehydroemetine, entecavir, glecaprevir and pibrentasvir, lamivudine, metadoxine, methionine, sofosbuvir, velpatasvir, and voxilaprevir, telbivudine, tenofovir, trientine, ursodeoxycholic acid, and the like.

In certain embodiments, user profile 118 is dynamic because at least part of the information that is stored in user profile 118 may be revised over time and/or new information may be added to user profile 118 by decision support engine 114 and/or application 106. Accordingly, information in user profile 118 stored in user database 110 provides an up-to-date repository of information related to a user.

User database 110, in some embodiments, refers to a storage server that operates in a public or private cloud. User database 110 may be implemented as any type of datastore, such as relational databases, non-relational databases, key-value datastores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 includes user profiles 118 associated with a plurality of users who similarly interact with application 106 executing on the display devices 107 of the other users. User profiles stored in user database 110 are accessible to not only application 106, but decision support engine 114, as well. User profiles in user database 110 may be accessible to application 106 and decision support engine 114 over one or more networks (not shown). As described above, decision support engine 114, and more specifically DAM 116 of decision support engine 114, can fetch inputs 128 from user database 110 and compute a plurality of metrics 130 which can then be stored as application data 126 in user profile 118.

In certain embodiments, user profiles 118 stored in user database 110 may also be stored in historical records database 112. User profiles 118 stored in historical records database 112 may provide a repository of up-to-date information and historical information for each user of application 106. Thus, historical records database 112 essentially provides all data related to each user of application 106, where data is stored according to an associated timestamp. The timestamp associated with information stored in historical records database 112 may identify, for example, when information related to a user has been obtained and/or updated.

Further, data stored in historical records database 112 may maintain time series data collected for users over a period of time such users use continuous analyte monitoring system 104 and application 106. For example, analyte data for a user who has used continuous analyte monitoring system 104 and application 106 for a period of five years to manage the user's liver condition may have time series analyte data associated with the user maintained over the five-year period.

Further, in certain embodiments, historical records database 112 may include data for one or more patients who are not users of continuous analyte monitoring system 104 and/or application 106. For example, historical records database 112 may include information (e.g., user profile(s)) related to one or more patients analyzed by, for example, a healthcare physician (or other known method), and not previously diagnosed with liver disease, as well as information (e.g., user profile(s)) related to one or more patients analyzed by, for example, a healthcare physician (or other known method) and were previously diagnosed with liver disease. Data stored in historical records database 112 may be referred to herein as population data.

Data related to each patient stored in historical records database 112 may provide time series data collected over the disease lifetime of the patient. For example, the data may include information about the patient prior to being diagnosed with liver disease and information associated with the patient during the lifetime of the disease, including information related to each stage of the liver disease as it progressed and/or regressed in the patient, as well as information related to other diseases, such as kidney disease or similar diseases that are co-morbid in relation to liver disease. Such information may indicate symptoms of the patient, physiological states of the patient, lactate levels of the patient, glucose levels of the patient, ketone levels of patient, potassium levels of the patient, states/conditions of one or more organs of the patient, habits of the patient (e.g., alcohol consumption, activity levels, food consumption, etc.), medication prescribed, etc. throughout the lifetime of the disease.

Although depicted as separate databases for conceptual clarity, in some embodiments, user database 110 and historical records database 112 may operate as a single database. That is, historical and current data related to users of continuous analyte monitoring system 104 and application 106, as well as historical data related to patients that were not previously users of continuous analyte monitoring system 104 and application 106, may be stored in a single database. The single database may be a storage server that operates in a public or private cloud.

As mentioned previously, decision support system 100 is configured to diagnose, stage, treat, and assess risks of liver disease of a user using continuous analyte monitoring system 104, including, at least, a continuous lactate sensor. In certain embodiments, to enable such diagnosis and staging, decision support engine 114 is configured to provide real-time and or non-real-time liver disease decision support to the user and or others, including but not limited, to healthcare providers, family members of the user, caregivers of the user, researchers, artificial intelligence (AI) engines, and/or other individuals, systems, and/or groups supporting care or learning from the data. In particular, decision support engine 114 may be used to collect information associated with a user in user profile 118 stored in user database 110, to perform analytics thereon for determining the probability of the presence and/or severity of liver disease for the user and providing one or more recommendations for treatment based, at least in part, on the determination. User profile 118 may be accessible to decision support engine 114 over one or more networks (not shown) for performing such analytics.

In certain embodiments, decision support engine 114 may utilize one or more trained machine learning models capable of performing analytics on information that decision support engine 114 has collected/received from user profile 118. In the illustrated embodiment of FIG. 1, decision support engine 114 may utilize trained machine learning model(s) provided by a training server system 140. Although depicted as a separate server for conceptual clarity, in some embodiments, training server system 140 and decision support engine 114 may operate as a single server. That is, the model may be trained and used by a single server, or may be trained by one or more servers and deployed for use on one or more other servers. In certain embodiments, the model may be trained on one or many virtual machines (VMs) running, at least partially, on one or many physical services in relational and or non-relational database formats.

Training server system 140 is configured to train the machine learning model(s) using training data, which may include data (e.g., from user profiles) associated one or more patients (e.g., users or non-users of continuous analyte monitoring system 104 and/or application 106) previously diagnosed with varying stages of liver disease, as well as patients not previously diagnosed with liver disease (e.g., healthy patients). The training data may be stored in historical records database 112 and may be accessible to training server system 140 over one or more networks (not shown) for training the machine learning model(s).

The training data refers to a dataset that has been featurized and labeled. For example, the dataset may include a plurality of data records, each including information corresponding to a different user profile stored in user database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model. As an illustrative example, each relevant characteristic of a user, which is reflected in a corresponding data record, may be a feature used in training the machine learning model. Such features may include age, gender, average change (e.g., average delta) in lactate clearance from a first timestamp to a second timestamp, average change (e.g., average delta) in liver disease diagnosis from a first timestamp to a subsequent timestamp, the derivative of the measured linear system of lactate measurement at a point in a specific timestamp and or the difference in derivatives to determine rates of change in the slope of the increase or decrease in value, etc. In addition, the data record is labeled with an indication as to the liver disease diagnosis, an assigned disease score, and/or an identified risk of liver disease, etc. associated with a patient of the user profile.

The model(s) are then trained by training server system 140 using the featurized and labeled training data. In particular, the features of each data record may be used as input into the machine learning model(s), and the generated output may be compared to label(s) associated with the corresponding data record. The model(s) may compute a loss based on the difference between the generated output and the provided label(s). This loss is then used to modify the internal parameters or weights of the model. By iteratively processing each data record corresponding to each historical patient, the model(s) may be iteratively refined to generate accurate predictions of liver disease risk, presence, progression, improvement, and severity in a patient.

As illustrated in FIG. 1, training server system 140 deploys these trained model(s) to decision support engine 114 for use during runtime. For example, decision support engine 114 may obtain user profile 118 associated with a user and stored in user database 110, use information in user profile 118 as input into the trained model(s), and output a prediction indicative of the presence and/or severity of liver disease for the user (e.g., shown as output 144 in FIG. 1). Output 144 generated by decision support engine 114 may also provide one or more recommendations for treatment based on the prediction. Output 144 may be provided to the user (e.g., through application 106), to a user's caretaker (e.g., a parent, a relative, a guardian, a teacher, a nurse, etc.), to a user's physician, or any other individual that has an interest in the wellbeing of the user for purposes of improving the user's health, such as, in some cases by effectuating the recommended treatment.

In certain embodiments, output 144 generated by decision support engine 114 may be stored in user profile 118. Output 144 may be indicative of the current health of a user, the state of a user's liver, and/or current treatment recommended to a user. Output 144 stored in user profile 118 may be continuously updated by decision support engine 114. Accordingly, previous diagnoses, originally stored as outputs 144 in user profile 118 in user database 110 and then passed to historical records database 112, may provide an indication of the progression of liver disease in a user over time, as well as provide an indication as to the effectiveness of different treatments recommended to a user to help stop progression of the disease.

In certain embodiments, a user's own historical data may be used by training server system 140 to train a personalized model for the user that provides decision support and insight around the user's liver disease. For example, a patient's historical data may be used as a baseline to indicate improvements or deterioration in the patient's liver function. As an illustrative example, a patient's data from 1 week ago; 2 weeks ago; 1 month ago; 6 months ago; or 1 year ago may be used as a baseline that can be compared with the patient's current data to identify whether the patient's liver function has improved or deteriorated. In certain embodiments, the model may further be able to predict or project out the patient's liver function or its future improvement/deterioration based on the user's recent pattern of data (e.g., exercise data, food consumption data, medication usage data, etc.).

In certain embodiments, the model may be trained to provide food, exercise, therapeutic intervention, medication type and dosage, and other types of decision support recommendations to help the user improve their liver function based on the user's historical data, including how different types of food and/or exercise impacted the user's liver function in the past. For example, where the model is trained to provide food and/or exercise recommendations, the model may be trained by training server system 140 based at least partially on historical glucose and lactate measurements after meals.

Generally, monitoring lactate levels of a user with liver disease over time as a measure of liver health may be desirable to provide positive or negative feedback to the user regarding specific lifestyle choices, including exercise, diet choices, medication type and dosage recommendations.

In certain embodiments, where the model is trained to provide therapeutic intervention recommendations, the model may be trained to provide recommendations on a specific type of therapy for the user based on the severity of liver disease of the user, historical lactate data, such as baseline lactate levels and lactate thresholds, as well as other analyte or non-analyte sensor data. Following the user implementing a therapy recommendation, the model may continue monitoring lactate data and other analyte and non-analyte sensor data to determine the impact of the recommended therapies on the user's liver health. Based on the data collected following the implementation of a therapy recommendation, the model may measure liver health over time to provide positive or negative feedback to the user regarding the therapeutic intervention.

In certain embodiments, the model may be trained by training server system 140 based on historical glucose and lactate to provide medication type and dosage recommendations. For example, the model may be trained to provide recommendations for medication type and dosage, based on the severity of liver disease for the user, historical lactate data, such as baseline lactate levels and lactate thresholds, as well as other historical analyte or non-analyte sensor data. Generally, monitoring lactate levels of the user over time may demonstrate the effect of a medication type or dosage on the user's liver health. Lactate levels over time may demonstrate, for example, that the user is sensitive to a particular type of statin and the user may be recommended to use an alternative statin.

In certain embodiments, the model may be trained to predict the underlying cause of certain improvements or deteriorations in the patient's liver function. For example, application 106 may display a user interface with a graph that shows the patient's liver functionality or a score thereof with trend lines and indicate, e.g., retrospectively, how the functionality suffered at certain points in time.

In certain embodiments where rule-based models are used for providing decision support, historical glucose and/or lactate measurements may be utilized to determine “healthy” and/or “unhealthy” thresholds or ranges for glucose and/or lactate levels post-consumption of a meal. Thereafter, the healthy and/or unhealthy thresholds or ranges for glucose and/or lactate may be utilized to notify the user about whether a meal was healthy or unhealthy for the user based on real-time measurements of glucose and/or lactate.

FIG. 2 is a diagram 200 conceptually illustrating an example continuous analyte monitoring system 104 including example continuous analyte sensor(s) with sensor electronics, in accordance with certain aspects of the present disclosure. For example, system 104 may be configured to continuously monitor one or more analytes of a user for which continuous analyte sensor(s) are attached, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 may be in wireless communication (e.g., directly or indirectly) with one or more of display devices 210, 220, 230, and 240. In certain embodiments, sensor electronics module 204 may also be in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 may comprise a sensor for detecting and/or measuring analyte(s). The continuous analyte sensor 202 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 202 may be configured to continuously measure analyte levels of a user using one or more measurement techniques, such as enzymatic, chemical, physical, electrochemical, spectrophotometric, polarimetric, calorimetric, iontophoretic, radiometric, immunochemical, and the like. In certain aspects the continuous analyte sensor 202 provides a data stream indicative of the concentration of one or more analytes in the user. The data stream may include raw data signals, which are then converted into a calibrated and/or filtered data stream used to provide estimated analyte value(s) to the user.

In certain embodiments, continuous analyte sensor 202 may be a multi-analyte sensor, configured to continuously measure multiple analytes in a user's body. For example, in certain embodiments, the continuous multi-analyte sensor 202 may be a single sensor configured to measure lactate, glucose, ketones, and/or potassium in the user's body.

In certain embodiments, one or more multi-analyte sensors may be used in combination with one or more single analyte sensors. As an illustrative example, a multi-analyte sensor may be configured to continuously measure lactate and glucose and may, in some cases, be used in combination with an analyte sensor configured to measure only ketones. Information from each of the multi-analyte sensor(s) and single analyte sensor(s) may be combined to provide liver disease decision support using methods described herein. In further embodiments, other non-contact and or periodic or semi-continuous, but temporally limited, measurements for physiological information may be integrated into the system such as by including weight scale information or non-contact heart rate monitoring from a sensor pad under the user while in a chair or bed, through an infra-red camera detecting temperature and/or blood flow patterns of the user, and/or through a visual camera with machine vision for height, weight, or other parameter estimation without physical contact.

In certain embodiments, sensor electronics module 204 includes electronic circuitry associated with measuring and processing the continuous analyte sensor data, including prospective algorithms associated with processing and calibration of the sensor data. Sensor electronics module 204 can be physically connected to continuous analyte sensor(s) 202 and can be integral with (non-releasably attached to) or releasably attachable to continuous analyte sensor(s) 202. Sensor electronics module 204 may include hardware, firmware, and/or software that enables measurement of levels of analyte(s) via a continuous analyte sensor(s) 202. For example, sensor electronics module 204 can include a potentiostat, a power source for providing power to the sensor, other components useful for signal processing and data storage, and a telemetry module for transmitting data from the sensor electronics module to one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which may be transmitted by sensor electronics module 204. Each of display devices 210, 220, 230, or 240 can include a display such as a touchscreen display 212, 222, 232, and/or 242 for displaying sensor data to a user and/or receiving inputs from the user. For example, a graphical user interface (GUI) may be presented to the user for such purposes. In some embodiments, the display devices may include other types of user interfaces such as a voice user interface instead of, or in addition to, a touchscreen display for communicating sensor data to the user of the display device and/or receiving user inputs. Display devices 210, 220, 230, and 240 may be examples of display device 107 illustrated in FIG. 1 used to display sensor data to a user of FIG. 1 and/or receive input from the user.

In some embodiments, one, some, or all of the display devices are configured to display or otherwise communicate the sensor data as it is communicated from the sensor electronics module (e.g., in a data package that is transmitted to respective display devices), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices may be configured for providing alerts/alarms based on the displayable sensor data. Display device 210 is an example of such a custom device. In some embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch, medical device 208 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data.

As mentioned, sensor electronics module 204 may be in communication with a medical device 208. Medical device 208 may be a passive device in some example embodiments of the disclosure. For example, medical device 208 may be an insulin pump for administering insulin to a user. For a variety of reasons, it may be desirable for such an insulin pump to receive and track glucose, lactate, and potassium values transmitted from continuous analyte monitoring system 104, where continuous analyte sensor 202 is configured to measure glucose, lactate, and/or potassium.

Further, as mentioned, sensor electronics module 204 may also be in communication with other non-analyte sensors 206. Non-analyte sensors 206 may include, but are not limited to, an altimeter sensor, an accelerometer sensor, a temperature sensor, a respiration rate sensor. Non-analyte sensors 206 may also include monitors such as heart rate monitors, blood pressure monitors, pulse oximeters, caloric intake, and medicament delivery devices. One or more of these non-analyte sensors 206 may provide data to decision support engine 114 described further below. In some aspects, a user may manually provide some of the data for processing by training server system 140 and/or decision support engine 114 of FIG. 1.

In certain embodiments, the non-analyte sensors 206 may be combined in any other configuration, such as, for example, combined with one or more continuous analyte sensors 202. As an illustrative example, a non-analyte sensor, e.g., a temperature sensor, may be combined with a continuous lactate sensor 202 to form a lactate/temperature sensor used to transmit sensor data to the sensor electronics module 204 using common communication circuitry. As another illustrative example, a non-analyte sensor, e.g., a temperature sensor, may be combined with a multi-analyte sensor 202 configured to measure lactate and glucose to form a lactate/glucose/temperature sensor used to transmit sensor data to the sensor electronics module 204 using common communication circuitry.

In certain embodiments, a wireless access point (WAP) may be used to couple one or more of continuous analyte monitoring system 104, the plurality of display devices, medical device(s) 208, and/or non-analyte sensor(s) 206 to one another. For example, WAP 138 may provide Wi-Fi and/or cellular connectivity among these devices. Near Field Communication (NFC) and or Bluetooth may also be used among devices depicted in diagram 200 of FIG. 2.

FIG. 3 illustrates example inputs and example metrics that are calculated based on the inputs for use by the decision support system of FIG. 1, according to some embodiments disclosed herein. In particular, FIG. 3 provides a more detailed illustration of example inputs and example metrics introduced in FIG. 1.

FIG. 3 illustrates example inputs 128 on the left, application 106 and DAM 116 in the middle, and metrics 130 on the right. In certain embodiments, each one of metrics 130 may correspond to one or more values, e.g., discrete numerical values, ranges, or qualitative values (high/medium/low, stable/unstable, etc.). Application 106 obtains inputs 128 through one or more channels (e.g., manual user input, sensors, other applications executing on display device 107, etc.). As mentioned previously, in certain embodiments, inputs 128 may be processed by DAM 113 to output metrics 130. Inputs 128 and metrics 130 may be used by training server system 140 to train and deploy one or more machine learning models for use by decision support engine 114 for diagnosing, staging, and assessing risks of liver disease.

In certain embodiments, starting with inputs 128, food consumption information may include information about one or more of meals, snacks, and/or beverages, such as one or more of the size, content (carbohydrate, fat, protein, etc.), sequence of consumption, and time of consumption. In certain embodiments, food consumption may be provided by a user through manual entry, by providing a photograph through an application that is configured to recognize food types and quantities, and/or by scanning a bar code or menu. In various examples, meal size may be manually entered as one or more of calories, quantity (“three cookies”), menu items (“Royale with Cheese”), and/or food exchanges (1 fruit, 1 dairy). In some examples, meal information may be received via a convenient user interface provided by application 106.

In certain embodiments, food consumption, food consumption information (the type of food (e.g., liquid or solid, snack or meal, etc.) and/or the composition of the food (e.g., carbohydrate, fat, protein, etc.)) may be determined automatically based on information provided by one or more sensors. Some example sensors may include body sound sensors (e.g., abdominal sounds may be used to detect the types of meal, e.g., liquid/solid food, snack/meal, etc.), radio-frequency sensors, cameras, hyperspectral cameras, and/or analyte (e.g., insulin, glucose, lactate, etc.) sensors to determine the type and/or composition of the food.

In certain embodiments, food consumption entered by a user may relate to lactate consumed by the user. Lactate for consumption may include any natural or designed food or beverage that contains lactate, such as a lactate drink, yogurt, or whole milk, for example. Lactate for consumption may also include any natural or designed food or beverage that is converted to lactate when it is absorbed by the body, such as a fructose drink, for example. As will be described in more detail with respect to metrics 130 computed by DAM 116, such lactate consumption may be used by DAM 116 to calculate lactate clearance rates of the user.

In certain embodiments, exercise information is also provided as an input. Exercise information may be any information surrounding activities requiring physical exertion by the user. For example, exercise information may range from information related to low intensity (e.g., walking) and high intensity (e.g., sprinting) physical exertion. In certain embodiments, exercise information may be provided, for example, by an accelerometer sensor on a wearable device such as a watch, fitness tracker, and/or patch. In certain embodiments, exercise information may also be provided through manual user input and/or through a surrogate sensor and prediction algorithm measuring changes to heart rate (or other cardiac metrics). When predicting that a user is exercising based on his/her sensor data, the user may be asked to confirm if exercise is occurring, what type of exercise, and or the level of strenuous exertion being used during the exercise over a specific period. This data may be used to train the system to learn about the user's exercise patterns to reduce the need for confirmation questions as time progresses and the training algorithm becomes optimized. Other analytes and sensor data may also be included in this training set, including analytes and other measured elements described herein including temporal elements such as time and day.

In certain embodiments, user statistics, such as one or more of age, height, weight, BMI, body composition (e.g., % body fat), stature, build, or other information may also be provided as an input. In certain embodiments, user statistics are provided through a user interface, by interfacing with an electronic source such as an electronic medical record, and/or from measurement devices. In certain embodiments, the measurement devices include one or more of a wireless, e.g., Bluetooth-enabled, weight scale and/or camera, which may, for example, communicate with the display device 107 to provide user data.

In certain embodiments, treatment/medication information is also provided as an input. Medication information may include information about the type, dosage, and/or timing of when one or more medications are to be taken by the user. Treatment information may include information regarding different lifestyle habits recommended by the user's physician. For example, the user's physician may recommend a user drink alcohol sparingly, exercise for a minimum of thirty minutes a day, or cut calories by 500 to 1,000 calories daily to improve liver health. In certain embodiments, treatment/medication information may be provided through manual user input.

In certain embodiments, analyte sensor data may also be provided as input, for example, through continuous analyte monitoring system 104. In certain embodiments, analyte sensor data may include lactate data (e.g., a user's lactate values) measured by at least a lactate sensor (or multi-analyte sensor) in continuous analyte monitoring system 104. In certain embodiments, analyte sensor data may include glucose data measured by at least a glucose sensor (or multi-analyte sensor) in continuous analyte monitoring system 104. In certain embodiments, analyte sensor data may include ketone data measured by at least a ketone sensor (or multi-analyte sensor) in continuous analyte monitoring system 104. In certain embodiments, analyte sensor data may include potassium data measured by at least a potassium sensor (or multi-analyte sensor) in continuous analyte monitoring system 104.

In certain embodiments, input may also be received from one or more non-analyte sensors, such as non-analyte sensors 206 described with respect to FIG. 2. Input from such non-analyte sensors 206 may include information related to a heart rate, a respiration rate, oxygen saturation, or a body temperature (e.g. to detect illness, physical activity, etc.) of a user. In certain embodiments, electromagnetic sensors may also detect low-power radio frequency (RF) fields emitted from objects or tools touching or near the object, which may provide information about user activity or location.

In certain embodiments, input received from non-analyte sensors may include input relating to a user's insulin delivery. In particular, input related to the user's insulin delivery may be received, via a wireless connection on a smart pen, via user input, and/or from an insulin pump. Insulin delivery information may include one or more of insulin volume, time of delivery, etc. Other parameters, such as insulin action time or duration of insulin action, may also be received as inputs.

In certain embodiments, time may also be provided as an input, such as time of day or time from a real-time clock. For example, in certain embodiments, input analyte data may be timestamped to indicate a date and time when the analyte measurement was taken for the user. In certain embodiments, however, time of day may not support a determination of whether the user is asleep or awake. When determining whether the user is asleep or awake, input received from non-analyte sensors (e.g., activity monitors), analyte sensors (e.g., lactate or glucose increase) and/or user input may inform a determination of whether the user is asleep or awake.

User input of any of the above-mentioned inputs 128 may be through a user interface, such a user interface of display device 107 of FIG. 1.

As described above, in certain embodiments, DAM 116 determines or computes the user's metrics 130 based on inputs 128. An example list of metrics 130 is shown in FIG. 3.

In certain embodiments, lactate levels may be determined from sensor data (e.g., lactate measurements obtained from a continuous lactate sensor of continuous analyte monitoring system 104). For example, lactate levels refer to time-stamped lactate measurements or values that are continuously generated and stored over time.

In certain embodiments, lactate production rates may be determined from sensor data (e.g., lactate measurements obtained from a continuous lactate sensor of continuous analyte monitoring system 104). In particular, lactate is produced from pyruvate (e.g., glucose is broken down to pyruvate) through enzyme lactate dehydrogenase during normal metabolism and exercise. In certain embodiments, a lactate production rate may be determined by assessing an increase in lactate levels over a specified amount of time. In certain embodiments, lactate production rates may be expressed as a percentage of a maximum heart rate (e.g., 85% of maximum heart rate) or a percentage of a maximum oxygen intake (e.g., 75%). In certain other embodiments, lactate production rates may be expressed as a function of accelerometer data. For example, accelerometer data may indicate a step rate of a user over time (e.g., increasing step rate shown by increasing accelerometer data and vice versa). Each of these step rates may correlate to a lactate level of a user a specified time. Thus, step rates analyzed over time (e.g., accelerometer data) and their corresponding lactate levels may provide information about a user's lactate production rate with respect to accelerometer data. DAM 116 may continuously, semi-continuously, or periodically measure a user's lactate production rate over time and store the lactate production rates with time-stamps in the user's profile 118. Lactate production rates may be time-stamped to allow for identifying a decrease or increase of the user's lactate production over time.

In certain embodiments, a lactate baseline may be determined from sensor data (e.g., lactate measurements obtained from a continuous lactate sensor of continuous analyte monitoring system 104). A lactate baseline represents a user's normal lactate levels during periods where fluctuations in lactate production is typically not expected. A user's baseline lactate is generally expected to remain constant over time, unless challenged through an action such as the consumption of lactate or lactate metabolic foods by the user or exercise by the user. Further, each user may have a different lactate baseline. In certain embodiments, a user's lactate baseline may be determined by calculating an average lactate levels over a specified amount of time where fluctuations are not expected. For example, the baseline lactate for a user may be determined over a period of time when the user is sleeping, sitting in a chair, or other periods of time where the user is sedentary and not consuming food or medication which would reduce or increase lactate levels. In certain embodiments, DAM 116 may continuously, semi-continuously, or periodically calculate a lactate baseline and time-stamp and store the corresponding information in the user's profile 118. In certain embodiments, DAM 116 may calculate the lactate baseline using lactate levels measured over a period of time where the user is sedentary, the user is not consuming lactate, and where no external conditions exist that would affect the lactate baseline exist. In certain other embodiments, DAM 116 may use lactate levels measured over a period of time where the user is, at least for a subset of the period of time, engaging in exercise and/or consuming lactate and/or a an external condition exists that would affect the lactate baseline. In this case, in some examples, DAM 116 may first identify which measured lactate values are to be used for calculating the baseline lactate by identifying lactate values that may have been affected by an external event, such the consumption of food, exercise, medication, or other perturbation that would disrupt the capture of a lactate baseline measurement. DAM 116 may exclude such measurements when calculating the lactate baseline of the user. In some other examples, DAM 116 may calculate the lactate baseline by first determining a percentage of the number of lactate values measured during this time period that represent the lowest lactate values measured. DAM 116 may then take an average of this percentage to determine the lactate baseline level.

In certain embodiments, a lactate clearance rate may be determined from sensor data (e.g., lactate measurements obtained from a continuous lactate sensor of continuous analyte monitoring system 104). In particular, a user's lactate clearance rate indicates the rate at which lactate metabolism is greater than lactate production. A lactate clearance rate may be indicative of liver function (e.g., the slope of a curve of lactate clearance may indicate liver function). In certain embodiments, the lactate clearance rate may be determined by calculating a slope between an initial lactate value (e.g., during a period of increased lactate levels) and a lactate baseline associated with the user. In certain embodiments, a lactate clearance rate may be calculated over time until the lactate levels of the user reach some value relative to the user's lactate baseline (e.g., 50% or 75% of lactate baseline). In certain embodiments, a lactate clearance rate may be calculated over time until the lactate levels of the user reach some value relative to a peak lactate level measured for the user at a previous time (e.g., lactate levels of the user reach 25%, 50%, and/or 75% of a peak lactate level of the user).

Further, monitoring lactate clearance rate after exercise or after a meal may demonstrate improvement or progression of liver disease. Because liver disease progression is typically slow, monitoring lactate clearance rates over time (e.g., after exercise or after a meal) and comparing current lactate clearance rates to past lactate clearance rates (after exercise or after a meal) may be helpful in determining disease progression over time. For example, if the lactate clearance rate becomes significantly delayed or trends in a worsening direction over time (e.g., lactate clearance rate is X at time Z and lactate clearance rate is X-Y at some future time Q), liver disease is progressing to a more severe state. In some embodiments, the decision support engine 114 would provide daily updates to users as lactate clearance rates are continuously monitored.

In certain embodiments, a lactate clearance rate may be expressed as a function of lactate half-life of a user. In particular, an inverse relationship exists between the lactate clearance rate and lactate half-life. In a diseased liver, the slope of lactate clearance is reduced as the calculated lactate half-life increases. As liver disease progresses, the slope of lactate clearance is further reduced and the calculated lactate half-life further increases. Thus, lactate half-life may be indicative of the lactate clearance rate of a user. Lactate clearance rates calculated over time may be time-stamped and stored in the user's profile 118.

In certain embodiments, a classifier may be used to determine whether data corresponds to a perturbation in the rate of increase or decrease in lactate followed by a sudden increase or decrease of the lactate of the user a different direction (e.g., resulting in what would otherwise be an unexpected change in the slope of lactate clearance of the user). These situations may include a user that was in a sedentary state and then started to exercise, stopped exercising for a brief period, and then began exercising again. In this case the rate of lactate production may not be constant, like the constant rate of clearance, as the increases in lactate generated during exercise would be proportional to the time dependency of the length of that exercise. Similarly, this may occur where a patient undergoing a lactate measurement on a fasted diet consumes an edible substance that increases the rate of lactate production in a consistent or inconsistent manner (e.g., depending on if the consumed substance is homogeneous or heterogeneous). In a heterogeneous substance, a change in lactate levels may be non-uniform as certain foods are digested at different rates based on the differences between sugars, proteins and fats of each of these foods. The data classifier system would aid in ruling in and/or ruling out relevant lactate production data (true rate increases) from lactate clearance data (true rate decreases). Further the data classifier system would aid in determining areas of variably elevated lactate or otherwise rapidly varied data in proportion to the adjustment in lactate production from consumption balance from overlapping increasing and decreasing signals.

In certain embodiments, a lactate trend may be determined based on lactate levels over certain periods of time. In certain embodiments, lactate trends may be determined based on lactate production rates over certain periods of time. In certain embodiments, lactate trends may be determined based on calculated lactate clearance rates over certain periods of time.

In certain embodiments, glucose levels may be determined from sensor data (e.g., blood glucose measurements obtained from a continuous lactate sensor of continuous analyte monitoring system 104). Elevated glucose levels may be used in combination with lactate levels to determine whether a user has consumed a meal. In order to more accurately determine the user has consumed a meal, elevated glucose levels and lactate levels may also be coupled with a body sound sensor, as previously described, to confirm the user has consumed a meal. Further, if glucose levels are coupled with data from an activity monitor, high lactate levels and high glucose levels may indicate high intensity exercise as opposed to consumption of a meal. Conversely, lower glucose levels in combination with high lactate levels may demonstrate that high lactate levels are due to conditions other than a meal (e.g., poor liver health, infection, or exercise).

In certain embodiments, a blood glucose trend may be determined based on glucose levels over a certain period of time.

In certain embodiments, insulin sensitivity may be determined using historical data, real-time data, or a combination thereof, and may, for example, be based upon one or more inputs 128, such as one or more of food consumption information, continuous analyte sensor data, non-analyte sensor data (e.g., insulin delivery information from an insulin device), etc. Insulin sensitivity refers to how responsive a user's cells are to insulin. Improving insulin sensitivity for a user may help to reduce insulin resistance in the user.

In certain embodiments, insulin on board may be determined using non-analyte sensor data input (e.g., insulin delivery information) and/or known or learned (e.g. from user data) insulin time action profiles, which may account for both basal metabolic rate (e.g., update of insulin to maintain operation of the body) and insulin usage driven by activity or food consumption.

In certain embodiments, insulin and or glucose sensor data (or derived values) may be used in combination with lactate sensor data in order to create a correction factor for certain activities (e.g., such as variations in anaerobic versus glucose metabolism rates). This may be especially important in people with diabetes on insulin with high levels of glycolysis leading to excessive pyruvate that can be converted to glucose, which often have high levels of circulating insulin. Therefore, there are complementary and inverse relationships of glucose, insulin, and/or lactate levels which may inform the health status of a patient or be useful in diagnosing the status of liver health.

Additionally, diagnosing the stage of liver disease over multiple sessions across many months may be a useful tool for sequentially determining the progression of the disease over time (e.g., as a user's actions or underlying health conditions result in the reversal or progression of the liver disease). Such disease progression/reversal information may be shared in a display to the patient, caregivers, family members, health insurance companies or other stakeholders interested in the patient's disease status over longer periods of time. Additionally, acute events such as liver decompensation and hypoglycemia in liver disease users, may be identified before severe acute symptoms become overwhelmingly debilitating by measuring rates of changes and absolute levels of insulin, glucose, and lactate in users.

In certain embodiments, ketone levels may be determined from sensor data (e.g., ketone measurements obtained from continuous analyte monitoring system 104). In certain embodiments, ketone levels may be expressed as a metric of whether or not the user is in ketosis. In particular, ketosis is a metabolic state in which there's a high concentration of ketones in the user's blood.

In certain embodiments, a ketone production rate may be determined from sensor data (e.g., ketone measurements obtained from a continuous ketone sensor of continuous analyte monitoring system 104). In particular, ketones (chemically known as ketone bodies) are byproducts of the breakdown of fatty acids. Glucose (e.g., blood sugar) is the preferred fuel source for many cells in the body; however, when there is limited access to glucose by the cells, fat may be broken down for fuel, thereby producing ketones as byproducts. In certain embodiments, a ketone production rate may be determined by assessing the increase in ketone levels over a specified amount of time.

In certain embodiments, potassium levels may be determined from sensor data (e.g., potassium measurements obtained from continuous analyte monitoring system 104).

In certain embodiments, health and sickness metrics may be determined, for example, based on one or more of user input (e.g., pregnancy information or known sickness information), from physiologic sensors (e.g., temperature), activity sensors, or a combination thereof. In certain embodiments, based on the values of the health and sickness metrics, for example, a user's state may be defined as being one or more of healthy, ill, rested, or exhausted.

In certain embodiments, disease stage metrics, such as for liver disease, may be determined, for example, based on one or more of user input or output provided by decision support engine 114 illustrated in FIG. 1. In certain embodiments, example disease stages for liver disease can include an inflammation stage (e.g., early stage where the user's liver is enlarged or inflamed), a fibrosis stage (e.g., stage with signs of scar tissue in the inflamed liver), a cirrhosis stage (e.g., stage with signs of severe scar tissue in the inflamed liver), an end-stage liver disease (ESLD). In certain embodiments, example disease stages may be represented as a NASH score, an NAFLD fibrosis score, a Child-Pugh score, a model for ESLD (MELD) score, a meta-analysis of histological data in viral hepatitis (METAVIR) score, and the like. Additionally, hepatocellular carcinoma may often be present throughout the later stages of cirrhosis and or ESLD.

In certain embodiments, decision support engine 114 may use a MELD score (or other liver disease metric/score) in combination with lactate data (or other analyte data) to predict liver disease progression and liver decompensation. Using the MELD score in combination with lactate data can be more effective or predictive than the MELD score alone or lactate data alone. For example, the MELD score alone may indicate liver disease, but rate of increase of lactate levels outside of meals or exercise correlate to the severity of the condition (e.g., liver damage or other systemic or organ damage). A high rate of change of lactate at rest when compared to past lactate rates of change may indicate a more severe liver dysfunction in combination with the patient's MELD score. In other cases, a high rate of change of lactate at rest when compared to past lactate rates of change may indicate other health conditions.

In certain embodiments, the meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first). A meal state metric may be determined based on information provided though user input or automatically based on information provided by one or more sensors (e.g., body sound sensors, as described above).

In certain embodiments, meal habits metrics are based on the content and the timing of a user's meals. For example, if a meal habit metric is on a scale of 0 to 1, the better/healthier meals the user eats the higher the meal habit metric of the user will be to 1, in an example. Also, the more the user's food consumption adheres to a certain time schedule, the closer their meal habit metric will be to 1, in the example. In certain embodiments, the meal habit metrics may indicate whether a user has been consistently participating in a ketogenic diet (e.g., a low-carb, moderate protein, and higher-fat diet) based on meals, snacks, or beverages consumed by the user over a certain period of time.

In certain embodiments, medication adherence is measured by one or more metrics that are indicative of how committed the user is towards their medication regimen. In certain embodiments, medication adherence metrics are calculated based on one or more of the timing of when the user takes medication (e.g., whether the user is on time or on schedule), the type of medication (e.g., is the user taking the right type of medication), and the dosage of the medication (e.g., is the user taking the right dosage). In certain embodiments, medication adherence of a user may be determined in a clinical trial where medication consumption and timing of such medication consumption is monitored.

In certain embodiments, the activity level metric may indicate the user's level of activity. In certain embodiments, the activity level metric be determined, for example based on input from an activity sensor or other physiologic sensors, such as non-analyte sensors 206. In certain embodiments, the activity level metric may be calculated by DAM 116 based on one or more of inputs 128, such as one or more of exercise information, non-analyte sensor data (e.g., accelerometer data), time, user input, etc. In certain embodiments, the activity level may be expressed as a step rate of the user. Activity level metrics may be time-stamped so that they can be correlated with the user's lactate levels at the same time.

In certain embodiments, exercise regimen metrics may indicate one or more of what type of activities the user engages in, the corresponding intensity of such activities, frequency the user engages in such activities, etc. In certain embodiments, exercise regimen metrics may be calculated based on one or more of non-analyte sensor data input (e.g., non-analyte sensor data input from an accelerometer, a heart rate monitor, a respiration rate sensor, etc.), calendar input, user input, etc.

In certain embodiments, metabolic rate is a metric that may indicate or include a basal metabolic rate (e.g., energy consumed at rest) and/or an active metabolism (e.g., energy consumed by activity, such as physical exertion). In some examples, basal metabolic rate and active metabolism may be tracked as separate outcome metrics. In certain embodiments, the metabolic rate may be calculated by DAM 113 based on one or more of inputs 128, such as one or more of exercise information, non-analyte sensor data, time, etc.

In certain embodiments, body temperature metrics may be calculated by DAM 113 based on inputs 128, and more specifically, non-analyte sensor data from a temperature sensor. In certain embodiments, heart rate metrics may be calculated by DAM 113 based on inputs 128, and more specifically, non-analyte sensor data from a heart rate sensor. In certain embodiments, respiratory metrics may be calculated by DAM 113 based on inputs 128, and more specifically, non-analyte sensor data from a respiratory rate sensor.

### Example Methods and Systems for Diagnosing, Staging, Treating, and Assessing Risks of Liver Disease Using Monitored Analyte Data

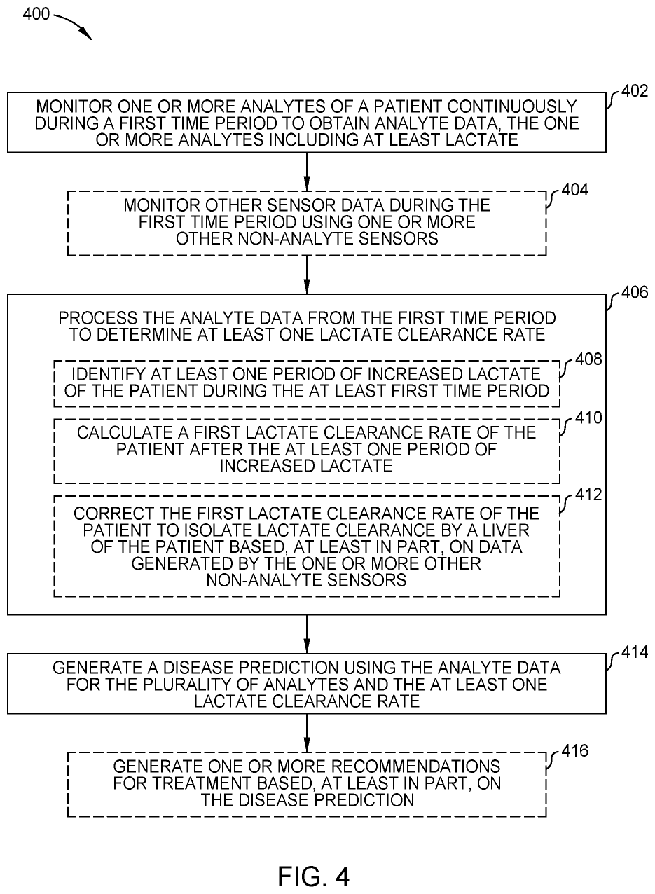

FIG. 4 is a flow diagram illustrating example method 400 for providing decision support using a continuous analyte sensor including, at least, a continuous lactate sensor, in accordance with certain example aspects of the present disclosure. For example, method 400 may be performed to provide decision support to a user, using a continuous analyte monitoring system 104 including, at least, a continuous lactate sensor 202, as illustrated in FIGS. 1 and 2. Method 400 may be performed by decision support system 100 to collect data, including for example, analyte data, patient information, and non-analyte sensor data mentioned above, to (1) automatically detect and classify abnormal liver conditions, (2) assess the presence and severity of liver disease, (3) risk stratify patients to identify those patients with a high risk of liver disease, (4) identify risks (e.g., mortality risk, liver cancer risk, etc.) associated with a current liver disease diagnosis, (5) make patient-specific treatment decisions or recommendations for liver disease, 6) provide information on the effect of an intervention (e.g., an effect of a lifestyle change of the patient, an effect of a surgical procedure, an effect of the patient taking new medication, etc.). In other words, the decision support system presented herein may offer information to direct and help improve care for patients with, or at risk, of liver disease. Method 400 is described below with reference to FIGS. 1 and 2 and their components.

At block 402, method 400 begins by continuously monitoring one or more analytes of a patient, such as user 102 illustrated in FIG. 1, during a first time period to obtain analyte data, the one or more analytes including at least lactate. Block 402 may be performed by continuous analyte monitoring system 104 illustrated in FIGS. 1 and 2, and more specifically, continuous analyte sensor(s) 202 illustrated in FIG. 2, in certain embodiments. For example, continuous analyte monitoring system 104 may comprise a continuous lactate sensor 202 configured to measure the user's lactate levels.

Lactate is the conjugate base of lactic acid. Lactate is produced from pyruvate (e.g., glucose is broken down to pyruvate) through enzyme lactate dehydrogenase during normal metabolism and exercise. Approximately up to 70% of lactate is metabolized by the liver. However, in very early liver disease, such as NAFLD, lactate metabolism is altered, which may lead to elevated levels of lactate in the body. Further, as the liver disease progresses, lactate production rates increase further, lactate metabolism becomes impaired, and lactate half-life increases. Lactate elevation may be caused by such increased production, decreased clearance, or both in combination. Accordingly, lactate may need to be continuously monitored to continually assess parameters such as lactate clearance rate (also indicative of lactate half-life), lactate levels, lactate production rates, and lactate baselines for diagnosing, staging, treating, and assessing risks of liver disease in real-time.

While the main analyte for measurement described herein is lactate, in certain embodiments, other analytes may be considered. In particular, combining lactate measurements with additional analyte data may help to further inform the analysis around diagnosing and staging liver disease. For example, monitoring additional types of analytes, such as glucose, ketones, and/or potassium measured by continuous analyte monitoring system 104, may provide additional insight into the liver disease diagnostics, and/or supplement information used to determine optimal treatment for preventing progression of the disease (and in some cases, for disease regression).

The additional insight gained from using a combination of analytes, and not just lactate, may increase the accuracy of liver disease diagnostics. For example, the probability of accurately diagnosing and/or staging liver disease may be a function of a number of analytes measured for a user. More specifically, in some examples, a probability of accurately staging liver disease using only lactate data (in addition to other non-analyte data) may be less than a probability of accurately staging liver disease using lactate and glucose data (in addition to other non-anlayte data), which may also be less than a probability of accurately staging liver disease using lactate, glucose, ketone, and potassium data (in addition to other non-anlayte data) for analysis.

In certain embodiments described herein, analyte combinations, e.g., measured and collected by one (e.g., multianalyte) or more sensors, for liver disease staging, include lactate and at least one of glucose, ketone, or potassium; however, other analyte combinations may be considered for diagnosing and staging liver disease.

For example, in certain embodiments, at block 402, continuous analyte monitoring system 104 may continuously monitor glucose levels of a user during a first time period. In some embodiments, glucose levels may be monitored in conjunction with or in lieu of other analytes (e.g., lactate, ketone, etc.). In such embodiments, the measured glucose concentrations may be used to further inform analysis for diagnosing and staging liver disease. In particular, in some cases, glucose levels are an indicator of a likelihood of developing insulin resistance and/or type II diabetes (T2D), and these pathologies increase the risk for liver disease.

For example, during digestion, foods that contain carbohydrates are converted into glucose, and the glucose is then sent into the bloodstream, causing a rise in blood glucose levels. This increase in blood glucose generally signals the pancreas to produce insulin. Insulin mediates precise regulation of glucose metabolism and plasma concentrations, not only by promoting glucose uptake by skeletal muscle, liver, and adipose tissue, but also by suppressing hepatic glucose production. Insulin plays an important role in lipid metabolism by combining with its receptor to promote fatty acid esterification, fatty acid storage in lipid droplets, and also inhibit lipolysis. However, in the context of insulin resistance, cells in the muscles, liver, and tissue do not respond well to insulin and cannot use glucose in the blood for energy. In response, the pancreas is stimulated to increase insulin secretion, leading to higher insulin levels in the liver as well as high concentrations of glucose in the blood. High concentrations of insulin may affect enzymes in the body leading to an increase in free fatty acids (FFAs) which may flow into the liver. An increase in FFAs may lead to an excessive amounts of fat stored in liver cells, and in some cases to NAFLD. In other words, patients who have insulin resistance, typically found in those with T2D, may be at a higher risk of developing NAFLD. Further, since T2D is a disease that may cause worsening of liver function, continuous glucose measurements may indicate the likelihood or state of T2D which may predict liver disease and/or NAFLD. Glucose metrics that may be used include glucose basic statistics (e.g., mean median, variation, inter-quartile range, etc.), glucose time-in-range, glucose peak metrics (e.g., peak counts, frequency, width, etc.), autocorrelation-related metrics (e.g., correlation coefficient, lag, etc.), and/or frequency-domain metrics (e.g., peak frequency, width of frequency peaks, etc.). Accordingly, monitoring glucose levels of a user may help inform the assessment of the likelihood of the user developing liver disease.

Further, liver disease and impaired liver function may result in frequent hyperglycemia, specifically after meals, as described above. Patients with liver disease may also experience nocturnal hypoglycemia as liver disease progresses. Thus, frequent postprandial hyperglycemia and nocturnal hypoglycemia, in combination with lactate measurements (e.g. higher baseline or resting lactate levels, postprandial lactate levels and impaired lactate clearance rates), may provide a more complete prediction of improvement or progression of liver disease, and/or may inform the assessment of the likelihood of the user developing liver disease.

In some embodiments, there are complementary and inverse relationships of glucose and lactate levels which may inform the health status of a patient or be useful in diagnosing the status of liver health. For example, in healthy users, lactate and glucose trends may be closely correlated (e.g., lactate and glucose levels may peak at similar times in response to events such as exercise or meal consumption). Thus, diverging lactate and glucose trends may indicate kidney or liver dysfunction in a user. For example, larger and/or delayed lactate peaks as compared to glucose peaks may indicate that the user has progressing liver disease or poor metabolic fitness, as the patient's body may not be able to clear substrates effectively and/or may not be able to switch between clearing lactate and glucose effectively.

In another example, at block 402, continuous analyte monitoring system 104 may continuously monitor ketone levels of the user, during a first time period. In some embodiments, ketone levels may be monitored in conjunction with or in lieu of other analytes (e.g., glucose, lactate, etc.). In such embodiments, the measured ketone concentrations may be used for diagnosing, staging, assessing risks of liver disease, and/or assessing different treatments for liver disease. For example, in some cases, ketone specific metrics may aid in the recommendation of a specific diet for the user diagnosed with liver disease, and further provide real-time feedback on the improvement of liver dysfunction after implementation of the recommended diet.

In some cases where the user has been previously diagnosed with NAFLD, measured ketone concentrations of the user, e.g., measured continuously using a continuous ketone sensor 202, may inform recommendation of treatment of the disease. For example, in some cases, based on the ketone concentrations of the user, a ketogenic diet may be recommended to the user. A ketogenic diet essentially aims to force the body into using a different type of fuel. Instead of relying on sugar (e.g., glucose) that comes from carbohydrates (such as grains, legumes, vegetables, and fruits), the ketogenic diet relies on ketone bodies, namely acetoacetate, acetone and β-hydroxybutyrate (βHB), a type of fuel that the liver produces from stored fat. A ketogenic diet may be used to put the user's body into ketosis (e.g., a metabolic state in which there's a high concentration of ketones in the blood) to ultimately reverse the effects of NAFLD. For a user diagnosed with fatty liver disease (e.g., NAFLD), eating more fat might seem counterintuitive; however, putting the user's body into ketosis triggers the body to start burning body fat, in addition to dietary fat. This may help to improve the health of the user's liver, as eventually, the user's body will begin eradicating the very problem that is causing the fatty liver. Improvements to the liver may, in this case, have a direct correlation to increased ketone concentrations in the user, e.g., due to implementation of the ketogenic diet.

In yet another example, at block 402, continuous analyte monitoring system 104 may continuously monitor a combination of two or more of lactate, glucose, and ketones of the user, during a first time period. In such embodiments, the measured concentrations are used to further inform the analysis around diagnosing and staging liver disease.

In particular, in certain embodiments, as mentioned previously, insulin mediates precise regulation of glucose metabolism and plasma concentrations by promoting glucose uptake by skeletal muscle, liver, and adipose tissue. Accordingly, where insulin is low, there is limited glucose for uptake by the skeletal muscle, liver, and adipose tissue. Such limited access to glucose, at least by the liver, causes the liver to instead break down fat for fuel (e.g., ketogenesis). Given ketones (e.g., ketone bodies) are byproducts of the breakdown of fatty acids, a high concentration of ketones in the blood may be expected when the liver breaks down such acids. However, where a user is diagnosed with liver disease, the liver may have an impaired ability to produce ketones (e.g., impaired ketogenesis); thus, the ketone concentrations may not be as high as expected.

In some cases, this may cause a user to believe they are in a healthy state, when in fact, the user is suffering from diabetic ketoacidosis (DKA) (e.g., where the bloodstream is flooded with extremely high levels of ketones). A user may not be aware that they have DKA given ketone concentrations expected for the user with DKA are being reduced by the impaired ability of the liver to produce such ketones (e.g., being masked by liver disease). Accordingly, low insulin and high blood glucose concentrations, combined with low ketone concentrations of a user, may be good indicator of liver impairment for informing diagnosis. This indication, combined with continuously measured lactate concentrations of a user, may help to increase the accuracy of predicting the presence and/or severity of liver disease in the user. Conversely, low blood glucose, in combination with high ketone concentrations, may indicate that the user is experiencing ketosis, which often results from a ketogenic diet. A ketogenic diet may improve liver health over time, and as a result, cause a user's lactate levels to also decrease. Therefore, a ketogenic diet may be recommended to users with liver disease to improve their liver health over time.

In another example, at block 402, continuous analyte monitoring system 104 may continuously monitor potassium levels of the user, during a first time period. In some embodiments, potassium levels may be monitored in conjunction with or in lieu of other analytes (e.g., glucose, lactate, ketone, etc.). Measuring potassium may help inform liver disease diagnosis and staging because reduced potassium excretion may be correlated with fatty liver disease. Alternatively, increased potassium excretion may be associated with chronic liver failure, and an increase in lactate may provide additional confirmation of a liver failure diagnosis, specifically in examples where the user is also suffering from an acute kidney injury. Acute kidney injury is a common complication for patients suffering from liver failure or cirrhosis. Thus, increasing lactate and potassium levels may indicate that the user is suffering from an acute kidney injury, which may be correlated to liver failure. However, if lactate levels increase while potassium levels remain stable, kidney injury is likely not a factor or cause of liver failure. In other examples, liver uptake of potassium in response to insulin may be impaired, causing hyperkalemia. In this case, hyperkalemia may occur independent of acute kidney injury. As such, combining potassium measurements with lactate, ketones, and/or glucose measurements may result in providing a more accurate diagnosis of liver disease.

In certain embodiments, AI models, such as machine learning models and/or algorithms may be used to provide real-time decision support for liver disease diagnosis and staging. In certain embodiments, such models may be configured to use input from one or more sensors measuring multiple analyte data to diagnose, stage, treat, and assess risks of liver disease. Accordingly, given the interaction of such comorbidities (e.g., as shown with respect to the example for a user with DKA and liver disease), parameters and/or thresholds of such algorithms and/or models may be altered based, at least in part, on a number of analytes being measured for input to reflect the knowledge attained from each of the other analytes being measured/morbidities associated with the additional analytes being measured.

In addition to continuously monitoring one or more analytes of a user during a first time period to obtain analyte data at block 402, optionally, in certain embodiments, at block 404, method 400 may also include monitoring other sensor data during the first time period using one or more other non-analyte sensors or devices. Block 404 may be performed by non-analyte sensors 206 and/or medical device 208 of FIG. 2, in certain embodiments.

As mentioned previously, non-analyte sensors and devices may include one or more of, but are not limited to, an insulin pump, a respiratory sensor, a heart rate monitor, an accelerometer, sensors or devices provided by display device 107 (e.g., accelerometer, camera, global positioning system (GPS), etc.) or any other sensors or devices that provide relevant information about the user. Metrics, such as metrics 130 illustrated in FIG. 3, may be calculated using measured data from each of these additional sensors. Further as illustrated in FIG. 3, metrics 130 calculated from non-analyte sensor or device data may include metabolic rate, body temperature, heart rate, respiratory rate, etc. In certain embodiments, described in more detail below, metrics 130 calculated from non-analyte sensor or device data may be used to further inform analysis for diagnosing and/or staging liver disease.

In certain embodiments, one or more non-analyte sensors and/or devices may be worn by a user to aid in the detection of periods of increased physical exertion by the user. Such non-analyte sensors and/or devices may include an accelerometer, an electrocardiogram (ECG) sensor, a blood pressure sensor, a heart rate monitor, and the like. In certain embodiments, measured and collected data from periods of increased physical exertion and periods of sedentary activity by the user may be used to analyze at least, kidney, heart, skeletal muscle, and/or liver function during each of these identified periods. In particular, approximately up to 70% of lactate is cleared by the liver with contributions from the kidneys, heart, and skeletal muscle during periods of sedentary activity by the user. The amount of lactate cleared by the liver may be lower than 70% during periods of physical exertion by the user (e.g., due to additional lactate being cleared the skeletal muscles and the heart). Accordingly, in certain embodiments, measured and collected data from periods of increased physical exertion and periods of sedentary activity by the user may be used to understand lactate clearance performed by the liver, kidney, heart, and/or muscle during each of these identified periods. As described in more detail below, understanding percentages of lactate clearance performed by different organs of the body may help to isolate lactate clearance performed by only the liver to better understand liver function, and any impairment where it may exist, to inform liver diagnostic and staging techniques described herein.

At block 406, method 400 continues by processing the analyte data from the first time period to determine at least one lactate clearance rate. Block 406, in certain embodiments, may be performed by decision support engine 114. As mentioned, even in very early liver disease, such as NAFLD, metabolism of lactate by the liver is impaired, and thus lactate has a longer half-life (as compared to lactate half-life in a healthy individual). Accordingly, lactate clearance rates (and lactate levels) may provide necessary information on liver health and/or a stage of liver disease. Note that although certain operations described herein with respect to method 400 involve calculating a lactate clearance rate (e.g., block 406) and/or using the lactate clearance rate for generating a disease prediction (e.g., block 414), instead of or in addition to a lactate clearance rate, one or more other lactate-derived metrics (e.g., lactate area under the curve, lactate baseline, lactate rate of change, post-prandial lactate, time above a specified lactate range (e.g., 2 mmol), time below a specified lactate range (e.g., 2 mmol), median lactate level, number of instances lactate is above or below a specified value, the amount of time lactate levels are within a certain range (e.g., 0.5 mmol to 1.5 mmol), the average or median rate of change of lactate over certain time periods (e.g., over a 24 hour period), the number of times lactate rates of change (absolute) are above a specified value, and/or information on these values when exercising or not exercising) may similarly be calculated and used to generate a disease prediction or generate a treatment recommendation as discussed relative to block 414 and block 416. Note that lactate area under the curve refers to the area on a graph between the lactate curve (e.g., representation of continuous lactate measurements depicted on the graph in relation to time) and the time axis, where time is measured on the X axis and lactate is measured on the Y axis.

Generally, the slope of lactate clearance is calculated by analysis of lactate measurements over time from a peak value, either (1) after exercise or (2) after consuming lactate, to a baseline value of lactate (e.g., which differs across different users and differs within a user based on different times of the day (e.g., morning versus afternoon versus nighttime baseline values of lactate for user)), some value relative to the baseline (e.g., 50% or 75% of baseline), or some value relative to the peak value (e.g., 25%, 50%, or 75% of the peak value). A lactate clearance rate calculated with the method above may correspond to an aggregation of lactate clearance performed by the liver, kidney, heart, and/or skeletal muscle.

Additionally, the level of lactate increase over time (e.g., change in peak value over time and/or lactate rate of change of increase), either (1) after exercise or (2) after consuming lactate (e.g., as part of a meal), may indicate liver dysfunction. For example, if a peak lactate value or lactate rate of change of increase after consuming lactate increases over time, the user may be experiencing worsening liver health and/or liver function.

To classify abnormal liver conditions, isolation of lactate clearance by the liver from the calculated lactate clearance may be desired; however liver lactate clearance isolation may present complex challenges. In particular, liver lactate clearance may be different for each user being analyzed, and further, may be different during different periods of physical exertion and/or inactivity of each user, given a user's kidney, heart, and liver also play an important role in clearing lactate in the body.

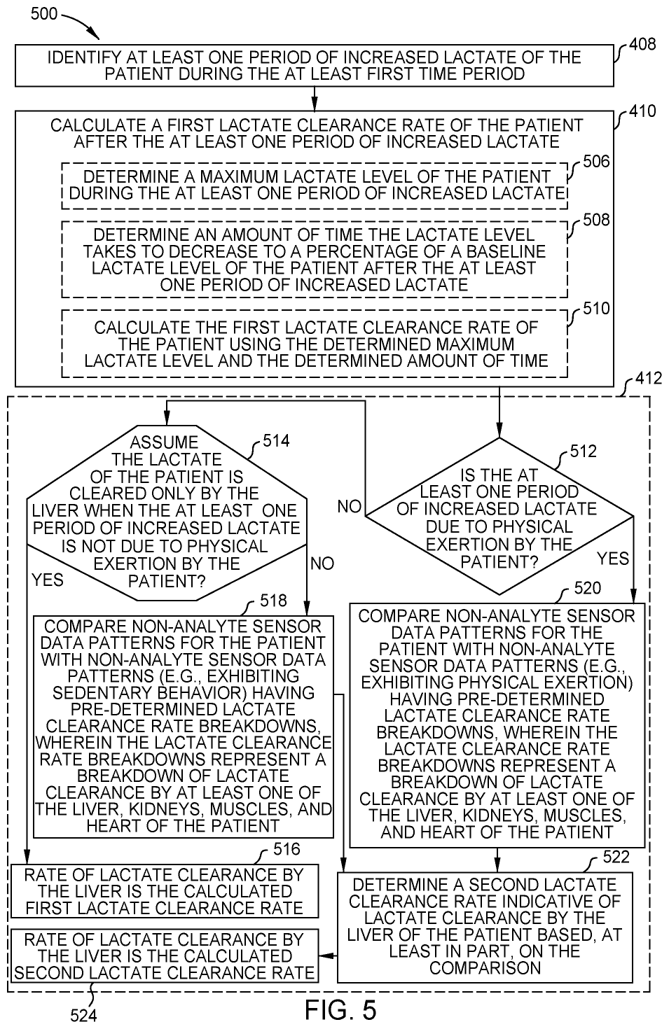

Techniques for isolating lactate clearance performed by the liver are provided herein. In particular, in certain embodiments, method 400 for determining at least one lactate clearance rate includes, at block 408, identifying at least one period of increased lactate of the user during the at least first time period, at block 410, calculating a first lactate clearance rate of the patient after the at least one period of increased lactate, and at block 412, correcting the first lactate clearance rate of the patient to isolate lactate clearance by a liver of the patient. Blocks 408, 410, and 412 of FIG. 4 may be better understood with reference to workflow 500 of FIG. 5.

FIG. 5 is an example workflow 500 for isolating a liver lactate clearance rate using at least, a continuous lactate monitor, according to certain embodiments of the present disclosure.

Workflow 500 of FIG. 5 may be performed by decision support system 100, including decision support engine 114. As shown in FIG. 5, workflow 500 begins at block 408 by decision support engine 114 identifying at least one period of increased lactate during the at least first time period when one or more of the user's analytes are continuously monitored to obtain analyte data. As an illustrative example, assuming the user is wearing a continuous analyte sensor 202 for continuously measuring lactate over a certain period, e.g., a 24-hour period, decision support engine 114 may identify periods of increased lactate concentration of the user during this 24-hour period. For this example, it may be determined that the user experienced peak lactate levels between 9 am-10 am and 1 pm-1:30 pm (determined based on the continuously measured lactate of the user).

At block 410, decision support engine 114 calculates a first lactate clearance rate for the user, after the at least one period of increased lactate. Using the above example, decision support system 100 calculates a first lactate clearance rate for the user after the identified period of high lactate levels during 9 am-10 am and another first lactate clearance rate for the user after the identified period of high lactate levels during 1 pm-1:30 pm.

In particular, at block 506, decision support engine 114 determines a maximum lactate level of the user, during the at least one period of increased lactate. At block 508, decision support system 100 determines an amount of time it takes for the maximum lactate level of the user to decrease to a percentage of a baseline lactate level of the user after the at least one period of increased lactate. In some cases, the baseline lactate level of the user may be a baseline lactate level of the user immediately preceding the increase in lactate levels of the user. In some cases, the baseline lactate level of the user may be a baseline lactate level of the user calculated as an average over a specified time range. For example, the baseline lactate levels of the user may be calculated as an average of morning lactate levels of the user, afternoon lactate levels of the user, evening lactate levels of the user, or etc. over one or more days. In certain embodiments, the baseline lactate level of the user may be a fasted baseline lactate level of the user. Although the example embodiment of FIG. 4 illustrates at block 508, decision support system 100 determining an amount of time it takes for the maximum lactate level of the user to decrease to a percentage of a baseline lactate level of the user, in certain other embodiments, decision support system 100 determines an amount of time it takes for the maximum lactate level of the user to decrease to a percentage of the maximum lactate level of the user (e.g., 25%, 50%, and/or 75% of the maximum lactate level). In certain embodiments, one or more of these slopes may be analyzed and compared for analysis. At block 510, decision support engine 114 calculates the first lactate clearance rate of the user, using the determined first lactate level, at block 506, and the determined amount of time, at block 508.

For example, at block 506, decision support engine 114 determines a maximum lactate level of the user during the identified periods of high lactate concentration throughout the 24-hour period, e.g., a first period between 9 am-10 am and a second period between 1 pm-1:30 pm, by analyzing lactate data collected during these periods. It may be assumed that, for this example, decision support engine 114 determines a maximum lactate level of 8 mmol/L between 9 am-10 am and a maximum lactate level of 5 mmol/L between 1 pm-1:30 pm.

At block 508, decision support engine 114 determines an amount of time it takes for the peak lactate value of each of the two identified periods to reduce to a baseline lactate level of the user. As mentioned with respect to FIG. 3, a baseline lactate level may be indicative of the user's normal lactate values while the user is at rest (e.g., sedentary). Assuming a baseline lactate level of the user is 2 mmol/L, decision support system 100 may determine an amount of time it takes measured lactate levels to reach 2 mmol/L after peak lactate concentrations of 8 mmol/L and 5 mmol/L.

At block 510, decision support engine 114 calculates a first lactate clearance rate for the user using the determined peak lactate concentration of 8 mmol/L, at block 506, and the amount of time determined at block 508. Further, decision support engine 114 calculates, for the second time period, a second lactate clearance rate of the user using the determined peak lactate concentration of 5 mmol/L, at block 506, and the amount of time determined at block 508. In other words, decision support engine 114 calculates a slope of lactate clearance over time from each of the identified peak lactate values.

In other embodiments, the first lactate clearance may be determined as part of a first lactate area under the curve for a first time period for the user using the determined peak lactate concentration of 8 mmol/L, at block 506, and the amount of time determined at block 508. The area under the curve may be calculated using the peak lactate concentration and the time from lactate baseline prior to the lactate peak to lactate return to baseline following the lactate peak. Lactate clearance may be useful in determining the rate of lactate return to baseline following the lactate peak and therefore useful in the area under the curve calculation. The area under the lactate curve of the first period would be compared to a lactate area under the curve of a second period. Further, decision support engine 114 calculates, for the second time period, a second lactate area under the curve of the user using the determined peak lactate concentration of 5 mmol/L, at block 506, and the amount of time determined at block 508. In other words, decision support engine 114 may calculate a slope of lactate area over time from each of the identified peak lactate values.

The first lactate clearance rate calculated at block 504 may be indicative of the aggregate lactate cleared by the liver, kidney, heart, and/or skeletal muscle of the user. To isolate lactate clearance performed by the liver of the user, decision support engine 114, at 412, corrects the first lactate clearance rate of the user to isolate lactate clearance by the user's liver. Therefore, the embodiments described herein provide a technical solution to the technical problem described above by correcting a lactate clearance rate to isolate lactate clearance by the user's liver. For example, decision support engine 114 performs steps at blocks 512-524 of FIG. 5 in order to correct a lactate clearance rate to accurately isolate the rate of lactate clearance by the liver and, therefore, more accurately generate a liver disease prediction.

At block 512, decision support engine 114 determines whether the identified at least one period of increased lactate was caused by physical exertion of the user. Lactic acid levels of a user increase when the user exercises thereby lowering the flow of blood and oxygen throughout the body, or when the user consumes lactate (e.g., yogurt or Cytomax, for example). A percentage of lactate clearance performed by each of the liver, kidney, heart, and/or skeletal muscle of the user may be different for each of a number of different scenarios. For example, in a scenario where the user has engaged in exercise (e.g., higher levels of physical exertion) and begins the cool off period (e.g., with mild exercise, such as walking, for example), muscles of the user may still be actively reducing lactate. Alternatively, in a scenario where a user is sedentary and consumes a lactate drink (e.g., milk), muscles of the user may not be actively reducing lactate. Thus, a larger percentage of the calculated lactate clearance may be cleared by the liver in scenarios where the user is sedentary and consumes a lactate drink, as opposed to scenarios where a user is in a cool off period after increased physical exertion (e.g., given muscles are performing a larger percentage of the lactate clearance in this scenario). Beyond the liver and skeletal muscles, in each of these scenarios, the heart and/or kidneys may also perform a percentage of the lactate clearance.

Accordingly, to differentiate liver lactate clearance from muscle lactate clearance, heart lactate clearance, and/or kidney lactate clearance, decision support system 100 may analyze non-analyte sensor data patterns of the user to identify periods of both physical exertion and inactivity to determine liver lactate clearance. In particular using mappings of non-analyte sensor data patterns to lactate clearance breakdowns (e.g., percentage of lactate clearance performed by the liver, skeletal muscles, heart, and/or kidney), decision support engine 114 may be able to better isolate liver lactate clearance from the first lactate clearance values calculated at block 512.

Such mappings may be pre-defined based on population data and/or the user's own data. The mappings may provide a mapping between non-analyte sensor data patterns, including accelerometer data patterns, respiratory sensor data patterns, and/or heart rate monitor data patterns, to lactate clearance breakdowns for each combination of these patterns.

For example, accelerometer data, heart rate data, and/or respiration data patterns demonstrating heightened values may be indicative of periods of increased physical exertion by a user. For periods of increased physical exertion, different accelerometer data, heart rate data, and/or respiration data patterns may be mapped to a percentage of lactate clearance (or lactate production) performed by each of the liver, heart, kidney, and skeletal muscle. Different activity types and/or different intensity levels may result in percentage variations for different non-analyte sensor data patterns.

Alternatively, accelerometer data, heart rate data, and/or respiration data patterns demonstrating lower values may be indicative of periods of minimal physical exertion by a user or periods of sedentary activity. For periods of minimal physical exertion, different accelerometer data, heart rate data, and/or respiration data patterns may be mapped to a percentage of lactate clearance performed by each of the liver, heart, kidney, and skeletal muscle. Different activity types and/or different levels of low physical exertion may result in percentage variations for different non-analyte sensor data patterns.

In certain embodiments, urine lactate levels may be used as input into decision support engine 114 to inform such mappings to more accurately predict lactate cleared by the kidneys. Both sedentary and activity data pattern mappings may be used to isolate lactate liver clearance by a user. In particular, based on different patterns of non-analyte sensor data, decision support system 100 may determine if a user is in an active state or a sedentary state during periods of increase lactate clearance.

At block 512, decision support engine 114 determines whether at least one period of increased lactate is due to physical exertion of the user. In some cases, decision support engine 114 may make this determination based on non-analyte sensor data patterns (e.g., including accelerometer data patterns, respiratory sensor data patterns, and/or heart rate monitor data patterns) indicating that the user is active or not active. In some other cases, decision support engine 114 may make this determination based on input provided by the user through application 106 (e.g., logging of exercise, logging of lactate consumption, logging of lactate infusion, etc.). Where at block 512, decision support engine 114 determines that the at least one period of increased lactate is not due to physical exertion of the user, decision support engine 114 determines that the increased lactate concentrations of the user during this identified period, are due, at least in part, to lactate consumption or lactate infusion.

In some cases, a user may consume lactate at his or her free will, while in other cases, the user may be directed to consume lactate to increase lactate levels of the user for measurement. Additionally, Pyruvate, Pyruvic Acid, and or other materials may be consumed to generate lactate in their breakdown. As mentioned, lactate for consumption may include any natural or designed food or beverage that contains lactate or other molecules designed to stimulate the production, metabolism, clearance, consumption, breakdown, or release of lactate and/or superseding and/or derivative metabolite to lactate in a measurable fashion. Additionally, synthetic lactate molecules or molecular mimics with enhanced diagnostic detecting elements (e.g., such as using radioactive or non-natural isotopes, enantiomers, quantum dot labeled probes, and/or other molecular differentiation techniques) may be used to differentiate the clearance of synthetic versus naturally generated lactate by measuring the lactate and/or the breakdown products directly, or indirectly, through inference of another analyte.

In some other cases, a user may be infused with lactate, consume lactate or consume a lactate producing meal (e.g., fructose) for measurement by continuous analyte monitoring system 104 to better determine lactate clearance rates of the user. For example, in some cases, lactate infusion may be used as a control scenario for determining lactate clearance of the user. This method may be an artificial way of challenging organs of the user by putting more lactate into the body to have direct knowledge of how much lactate is being input (e.g., because an operator of the lactate infusion pump is in control of the infusion and the pump) for clearance. For example, this process may involve infusing lactate into the user's body at a rate of 10 mL/hr, 20 mL/hr, or 30 mL/hr until the lactate reaches a control amount and allow for the organs of the user to clear the infused lactate. In certain embodiments, instead of lactate infusion, lactate may be consumed. Oral lactate consumption undergoes first-pass liver metabolism and, therefore, the peak level of lactate following consumption of a standardized beverage or meal may be a way to isolate the liver and evaluate its health, as long as the subject is not exercising during lactate consumption. If there is impaired lactate metabolism in the liver, a patient with liver disease may show higher rate of increase of lactate, longer duration of peak lactate levels, slower lactate clearance and/or higher than expected values following consumption of a lactate meal/drink.

Continuing with the example provided above, based on either user input or analyzing one or more patterns of data from one or more non-analyte sensors, at block 512, decision support engine 114 determines that, during the identified first time period of increased lactate levels (e.g., between 9 am-10 am), the maximum lactate level of 8 mmol/L was achieved over a period of physical exertion by the user. Additionally, decision support engine 114 determines that, during the identified second time period of increased lactate levels of the user (e.g., between 1 pm-1:30 pm), the maximum lactate level of 5 mmol/L was achieved over a period of inactivity (or no physical exertion) by the user.

Because, at block 512, decision support engine 114 determines the second time period of increased lactate levels of the user (e.g., between 1 pm-1:30 pm) is not due to physical exertion, at block 514, decision support engine 114 determines whether to assume the lactate in the user's body is cleared only by the liver.

In particular, in certain embodiments, decision support engine 114 may be configured to assume that where the user is determined to be sedentary (e.g., no physical activity), the lactate clearance rate calculated at block 504 is indicative of mainly liver lactate clearance. In other words, decision support engine 114 may assume that while sedentary, no other organs are performing a significant amount of the clearance; thus, no correction is necessary to isolate liver lactate clearance from the lactate clearance rate calculated at block 504. For example, decision support engine 114 may assume that when a user consumes a lactate drink, the user's muscles may not be actively producing lactate; thus, no correction is necessary to isolate liver lactate clearance from the lactate clearance rate calculated at block 504. Accordingly, at block 516, lactate clearance by the user's liver is determined to be the first lactate clearance rate (e.g., calculated at block 410). As described in more detail below, decision support engine 114 may use this first lactate clearance as a metric for predicting the presence and/or severity of liver disease in the user.

In certain other embodiments, decision support engine 114 may be configured to conclude that, where the user is determined to be sedentary (e.g., physical activity), the lactate clearance rate calculated at block 410 is indicative of lactate clearance performed by the liver as well as other organs. In other words, although the user is determined to be inactive during the period of high lactate concentrations, the sedentary lactate clearance rate may not represent 100% liver lactate clearance.

Accordingly, at block 518, decision support engine 114 compares the first lactate clearance rate calculated for the at least one period of increased lactate (e.g., at block 410) to one or more lactate clearance rates calculated for one or more periods of sedentary behavior by the user, wherein each of the one or more other lactate clearance rates represents an aggregation of lactate clearance by at least one of the liver, kidneys, muscles, and/or the heart. In particular, decision support engine 114 may compare the user's non-analyte sensor data patterns with mappings of non-analyte sensor data patterns (e.g., exhibiting sedentary behavior) to pre-determined lactate clearance rate breakdowns (e.g., percentage of lactate clearance performed by the liver, skeletal muscles, heart, and/or kidney). Based on the comparison, decision support engine 114 may identify a non-analyte sensor data pattern in the mappings that most closely resembles the user's current non-analyte sensor data pattern (e.g., representative of sedentary activity). The identified non-analyte sensor data pattern maps to a pre-determined lactate clearance rate breakdown, which decision support system 100 identifies as the user's lactate clearance rate breakdown.

As an illustrative example, collected non-analyte data for a user may include accelerometer data and respiratory data where the user is using an accelerometer and a respiratory monitor. The accelerometer data collected for the user may represent a first pattern, X, while the respiratory data collected for the user may represent a second pattern, Y. Decision support engine 114 may compare these two patterns to mappings of other non-analyte sensor data patterns. A first non-analyte sensor data pattern may include an accelerometer data pattern A and a respiratory data pattern B. It may have been previously determined that for this first non-analyte sensor data pattern, the liver is clearing 70% of lactate in the body while the kidneys and muscles are clearing the remaining 30%. A second non-analyte sensor data pattern may include an accelerometer data pattern X and a respiratory data pattern Y. It may have been previously determined that for this second non-analyte sensor data pattern, the liver is clearing 60% and the kidneys and muscles clearing the remaining 40%. During comparison, decision support engine 114 may determine accelerometer data pattern X and respiratory data pattern Y most closely resemble the second non-analyte sensor data pattern. Accordingly, decision support engine 114 may determine that, based on the pre-determined lactate clearance rate breakdown for the second non-analyte sensor data pattern, 60% of the lactate cleared is being cleared by the liver.

At block 522, decision support engine 114 determines a second lactate clearance rate indicative of lactate clearance by only the liver based, at least in part, on the comparison performed at block 518. For example, where a similar non-analyte data pattern is located in the mappings, decision support engine 114 may determine based on the user's non-analyte data, the user's liver is likely clearing only 70% of the lactate in the user's body. Accordingly, decision support engine 114 may adjust the lactate clearance rate calculated at block 410 based, at least in part, on the determination that the liver is likely only contributing to 70% of the calculated clearance. Accordingly, at block 524, the rate of lactate clearance by the user's liver is determined to be the second lactate clearance rate (e.g., calculated at block 522). As described in more detail below, decision support engine 114 may use this second lactate clearance as a metric for predicting the presence and/or severity of liver disease in the user.

Alternatively, returning to block 512, having determined that the at least one period of increased lactate is due to physical exertion, decision support engine 114 then determines that the increased lactate concentrations of the user during this identified period, are due, at least in part, to increased activity. For example, because at block 512, decision support engine 114 determines the first time period of increased lactate levels (e.g., between 9 am-10 am) is due to physical exertion, decision support engine 114 may assume that during the hours of 9 am and 10 am the user was engaging in some physical activity or exercise.

In some cases, the user may exercise at his or her free will, while in other cases, the user may be directed to engage in some form of exercise or physical exertion (also referred to as exercise-induced lactate analysis). For example, one concept for measuring lactate levels is to have the user exercise at an intensity such that the user's lactate level increases to a certain level, e.g., between 4-10 mmol/L, or reaches the user's lactate threshold, for example. Once this level is achieved, exercise may be stopped, and lactate clearance may be measured. The level of lactate may be held at a certain value, e.g., such as 5 mmol/L, for some period of time (for example 5-10 minutes) prior to stopping exercise.

Continuing with the example provided above, because, at block 512, decision support engine 114 determines that the maximum lactate level of 8 mmol/L for the user was achieved during a period of physical exertion, at block 520, decision support engine 114 compares non-analyte sensor data patterns for the user with mappings of non-analyte sensor data patterns (e.g., exhibiting physical exertion) to pre-determined lactate clearance rate breakdowns (e.g., percentage of lactate clearance performed by the liver, skeletal muscles, heart, and/or kidney). Decision support engine 114 may perform such a comparison to identify a non-analyte sensor data pattern that most closely resembles or is related to the user's current non-analyte sensor data pattern (e.g., representative of physical exertion) The identified non-analyte sensor data pattern maps to a pre-determined lactate clearance rate breakdown, based on which decision support engine 114 identifies as the user's lactate clearance rate being performed by the user given the current activity level of the user.

At block 522, decision support engine 114 determines a second lactate clearance rate indicative of lactate clearance only by the liver based, at least in part, on the comparison at block 520. For example, where a similar non-analyte data pattern is located, decision support engine 114 may determine based on the user's non-analyte data the user's liver is likely clearing only 50% of the lactate in the user's body. Accordingly, decision support engine 114 may adjust the lactate clearance rate calculated at block 504 based, at least in part, on the determination that the liver is likely only contributing to 50% of the calculated clearance. Accordingly, at block 524, the rate of lactate clearance by the user's liver is determined to be the second lactate clearance rate (e.g., calculated at block 522). As described in more detail below, decision support engine 114 may use this second lactate clearance as a metric for predicting the presence and/or severity of liver disease in the user.

In certain embodiments, where more than one period of increased lactate levels are identified at block 502 and analyzed to determine multiple liver lactate clearance rates, each liver lactate clearance rate that is calculated (and corrected, in some cases) may be used independently as an input to diagnose and/or stage the user's liver disease. In certain other embodiments, an average liver lactate clearance rate may be determined based on one or more of the calculated liver lactate clearance rates, and the average calculated liver lactate clearance rate may be used independently as an input to diagnose and/or stage the user's liver disease.

Referring back to FIG. 4, method 400 continues at block 414 by decision support system 100 generating a disease prediction using the analyte data associated with the one or more analytes and the at least one lactate clearance rate (e.g., determined and, in some cases, corrected according to workflow 500 of FIG. 5). Block 414 may be performed by decision support engine 114 illustrated in FIG. 1, in certain embodiments.

Different methods for generating a disease prediction may be used by decision support engine 114. In particular, in certain embodiments, decision support engine 114 may use a rule-based model to provide real-time decision support for liver disease diagnosis and staging. Rule-based models involve using a set of rules for manipulating and/or analyzing data. These rules are sometimes referred to as ‘If statements’ as they tend to follow the line of ‘If X happens then do Y’. In particular, decision support engine 114 may apply rule-statements (e.g., if, then statements) to assess the presence and severity of liver disease in a user, perform liver disease risk stratification for a user, and/or identify risks (e.g., mortality risk, liver cancer risk, etc.) associated with a current liver disease diagnosis of the user.

For example, a first rule may be “If a patient's liver lactate clearance rate falls between X & Y, then the patient has liver disease stage 1 of a particular scoring system (or that corresponds to a first METAVIR score)” while a second rule may be “If liver lactate clearance falls between Y & Z, then the patient has liver disease stage 2 of the particular scoring system (or that corresponds to a second METAVIR score)”. The determined liver lactate clearance (e.g., determined at block 408) may be applied against these predefined rules to stage liver disease.

Such rules may be defined and maintained by decision support engine 114 in a reference library. For example, the reference library may maintain ranges of liver lactate clearance rates which may be mapped to liver disease stages. In certain embodiments, such rules may be determined based on training server system 140 analyzing historical patient records from historical records database 112.

In some cases, the reference library may become very granular. For example, other factors may be used in the reference library to create such “rules”. Other factors may include gender, age, diet, disease history, family disease history, body mass index (BMI), etc. Increased granularity may provide more accurate outputs. As an example, including age ranges in the rule-based approach, e.g., used by decision support engine 114, may help inform differences in lactate clearance rates such that liver disease prediction, staging, diagnosis, etc. by decision support engine 114 is more accurate. For example, the average liver lactate clearance rate of a teenager (e.g., 13-17 years old) may be different from the average lactate clearance rate of a middle-age adult (e.g., 30-50 years old); thus, age might be an important factor to analyze in the rule-based approach to better predict and stage liver disease in users.

In certain embodiments, as an alternative to using a rule-based model, AI models, such as machine learning models may be used to provide real-time decision support for liver disease diagnosis and staging. In certain embodiments, decision support engine 114 may deploy one or more of these machine learning models for performing diagnosis, staging, and risk stratification of liver disease in a user. Risk stratification may refer to the process of assigning a health risk status to a user, and using the risk status assigned to the user to direct and improve care.

In particular, decision support engine 114 may obtain information from a user profile 118 associated with a user, stored in user database 110, featurize information for the user stored in user profile 118 into one or more features, and use these features as input into such models. Alternatively, information provided by the user's profile 118 may be featurized by another entity and the features may then be provided to decision support engine 114 to be used as input into the ML models. In machine learning, a feature is an individual measurable property or characteristic that is informative for analysis. In certain embodiments, features associated with the user may be used as input into one or more of the models to assess the presence and severity of liver disease in the user. In certain embodiments, features associated with the user may be used as input into one or more of the models to risk stratify the user to identify whether there is a high or low risk of the user developing liver disease. In certain embodiments, features associated with the user may be used as input into one or more of the models to identify risks (e.g., mortality risk, liver cancer risk, etc.) associated with a current liver disease diagnosis of the user.

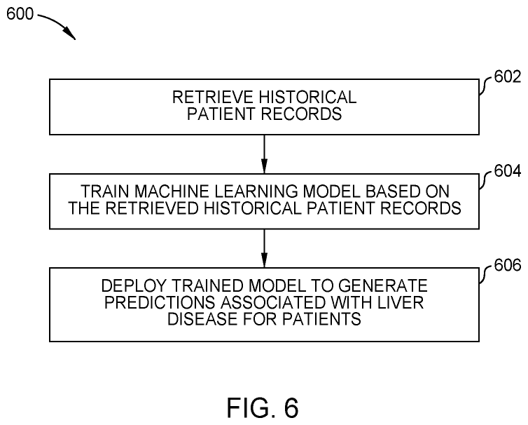

In certain embodiments, features associated with the user may be used as input into one or more of the models to perform any combination of the above-mentioned functions. Details associated with how one or more machine learning models can be trained to provide real-time decision support for liver disease diagnosis and staging are further discussed in relation to FIG. 6.

As mentioned, in certain embodiments, other analyte data, in addition to lactate, may be used by decision support engine 114 to generate a disease prediction for a user, at block 414. Analyte data, including lactate and glucose data, lactate and ketone data, lactate and potassium data, or lactate, glucose, potassium, and ketone data (e.g., from measurements by continuous analyte monitoring system 104), may be used as input into such machine learning models and/or rule-based models to predict the presence and severity of liver disease of a user. Decision support engine 114 may use the machine learning models and/or the rule-based models to generate a disease prediction based on continuous analysis of data (e.g., analyte data and, in some cases, non-analyte data) for the user collected over various time periods. Analysis of data collected for the user over various time periods may provide insight into whether the health and/or a disease of the user is improving or deteriorating. For example, a user previously diagnosed with liver disease using the models discussed herein may continue to be constantly monitored (e.g., continuously collect for the user) to determine whether the disease is getting worse or better, etc. As an example, comparison of lactate clearance rates, peak lactate levels, baseline lactate levels, and/or lactate production rates (e.g., after the consumption of lactate) for a user over multiple months may be indicative of the user's disease progression.

In some cases, method 400 continues at block 416 by decision support engine 114 generating one or more recommendations for treatment, based, at least in part, on the disease prediction generated at block 414. In particular, decision support engine 114 makes liver disease treatment decisions or recommendations for the user. Treatment recommendations may include recommendations for lifestyle modification and/or one or more drugs to prescribe, titrate, or avoid use by the user. Decision support engine 114 may output such recommendations for treatment to the user (e.g., through application 106).

As an illustrative example, in some cases, decision support engine 114 may determine liver disease in a user is progressing and correlate such progression to a drug previously prescribed for the user. For example, liver lactate clearance rates may be severely impaired where there is an acute liver toxicity from a specific drug. Accordingly, based on input medication consumption information for the user (in combination with other factors), decision support engine 114 may determine such progression of the disease is attributed to one or more medications previously prescribed to the user. Accordingly, in certain embodiments, decision support engine 114, at block 416, may recommend the user stop taking the previously prescribed medication, and in some cases, recommend an alternative medication for consumption by the user. In certain other embodiments, decision support engine 114, at block 416, may recommend the user take a lower dosage of the previously prescribed medication. In certain embodiments, decision support engine 114 may recommend titration of the dosage of the previously prescribed medication to determine an ideal dosage for the user (e.g., while monitoring liver health of the user).

In certain embodiments, machine learning models deployed by decision support engine 114 include one or more models trained by training server system 140, as illustrated in FIG. 1. FIG. 6 describes in further detail techniques for training the machine learning model(s) deployed by decision support engine 114 for diagnosis, staging, and risk stratifying liver disease of a patient, e.g., a user, according to certain embodiments of the present disclosure.

FIG. 6 is a flow diagram depicting a method 600 for training machine learning models to provide a prediction of liver disease diagnosis, according to certain embodiments of the present disclosure. In certain embodiments, the method 600 is used to train models to evaluate the presence and/or severity of liver disease in a patient, e.g., a user illustrated in FIG. 1.

Method 600 begins, at block 602, by a training server system, such as training server system 140 illustrated in FIG. 1, retrieving data from a historical records database, such as historical records database 112 illustrated in FIG. 1. As mentioned herein, historical records database 112 may provide a repository of up-to-date information and historical information for users of a continuous analyte monitoring system and connected mobile health application, such as users of continuous analyte monitoring system 104 and application 106 illustrated in FIG. 1, as well as data for one or more patients who are not, or were not previously, users of continuous analyte monitoring system 104 and/or application 106. In certain embodiments, historical records database 112 may include one or more data sets of historical patients with no liver disease or varying stages of liver disease.

Retrieval of data from historical records database 112 by training server system 140, at block 602, may include the retrieval of all, or any subset of, information maintained by historical records database 112. For example, where historical records database 112 stores information for 100,000 patients (e.g., non-users and users of continuous analyte monitoring system 104 and application 106), data retrieved by training server system 140 to train one or more machine learning models may include information for all 100,000 patients or only a subset of the data for those patients, e.g., data associated with only 50,000 patients or only data from the last ten years.

As an illustrative example, integrating with on premises or cloud based medical record databases through Fast Healthcare Interoperability Resources (FHIR), web application programming interfaces (APIs), Health Level 7 (HL7), and or other computer interface language may enable aggregation of healthcare historical records for baseline assessment in addition to the aggregation of de-identifiable patient data from a cloud based repository.

As an illustrative example, at block 602, training server system 140 may retrieve information for 100,000 patients with varying stages of liver disease stored in historical records database 112 to train a model to predict the risk, presence, and/or severity of liver disease in a user. Each of the 100,000 patients may have a corresponding data record (e.g., based on their corresponding user profile)), stored in historical records database 112. Each user profile 118 may include information, such as information discussed with respect to FIG. 3.

The training server system 140 then uses information in each of the records to train an artificial intelligence or ML model (for simplicity referred to as “ML model” herein). Examples of types of information included in a patient's user profile were provided above. The information in each of these records may be featurized (e.g., manually or by training server system 140), resulting in features that can be used as input features for training the ML model. For example, a patient record may include or be used to generate features related to an age of a patient, a gender of the patient, an occupation of the patient, lactate clearance rates, lactate area under the curve, an average change (e.g., average delta) in lactate clearance from a first timestamp to a subsequent timestamp for the patient, other lactate metrics described herein, an average change (e.g., average delta) in liver disease diagnosis from a first timestamp to a subsequent timestamp for the patient, and/or any other data points in the patient record (e.g., inputs 128, metrics 130, etc.). Features used to train the machine learning model(s) may vary in different embodiments.

In certain embodiments, each historical patient record retrieved from historical records database 112 is further associated with a label indicating whether the patient was healthy or experienced some variation of liver disease, a previously determined liver disease diagnosis and/or stage of liver disease for the patient, a previously assigned Child-Pugh score, MELD score, and/or METAVIR score, an NAFLD score, a NASH score, risk assessment, treatment recommendations, and similar metrics. What the record is labeled with would depend on what the model is being trained to predict.

At block 604, method 600 continues by training server system 140 training one or more machine learning models based on the features and labels associated with the historical patient records. In some embodiments, the training server does so by providing the features as input into a model. This model may be a new model initialized with random weights and parameters, or may be partially or fully pre-trained (e.g., based on prior training rounds). Based on the input features, the model-in-training generates some output. In certain embodiments, the output may indicate whether the patient was healthy or experienced some variation of liver disease, a liver disease diagnosis and/or liver disease stage for the patient, a Child-Pugh score, an MELD score, a METAVIR score, an NAFLD score, a NASH score, a risk assessment, a treatment recommendation, or similar outputs. Note that the output could be in the form of a likelihood, a classification, and/or other types of output.

In certain embodiments, training server system 140 compares this generated output with the actual label associated with the corresponding historical patient record to compute a loss based on the difference between the actual result and the generated result. This loss is then used to refine one or more internal weights and parameters of the model (e.g., via backpropagation) such that the model learns to predict the presence and/or severity of liver disease (or its recommended treatments) more accurately.

One of a variety of machine learning algorithms may be used for training the model(s) described above. For example, one of a supervised learning algorithm, a neural network algorithm, a deep neural network algorithm, a deep learning algorithm, etc. may be used.

At block 606, training server system 140 deploys the trained model(s) to make predictions associated with liver disease during runtime. In some embodiments, this includes transmitting some indication of the trained model(s) (e.g., a weights vector) that can be used to instantiate the model(s) on another device. For example, training server system 140 may transmit the weights of the trained model(s) to decision support engine 114. The model(s) can then be used to assess, in real-time, the presence and/or severity of liver disease of a user using application 106, provide treatment recommendations, and/or make other types of predictions discussed above. In certain embodiments, the training server system 140 may continue to train the model(s) in an “online” manner by using input features and labels associated with new patient records.

Further, similar methods for training illustrated in FIG. 6 using historical patient records may also be used to train models using patient-specific records to create more personalized models for making predictions associated with liver disease. For example, a model trained using historical patient records that is deployed for a particular user, may be further re-trained after deployment. For example, the model may be re-trained after the model is deployed for a specific patient to create a more personalized model for the patient. The more personalized model may be able to more accurately make liver disease-related predictions for the patient based on the patient's own data (as opposed to only historical patient record data), including the patient's own inputs 128 and metrics 130.

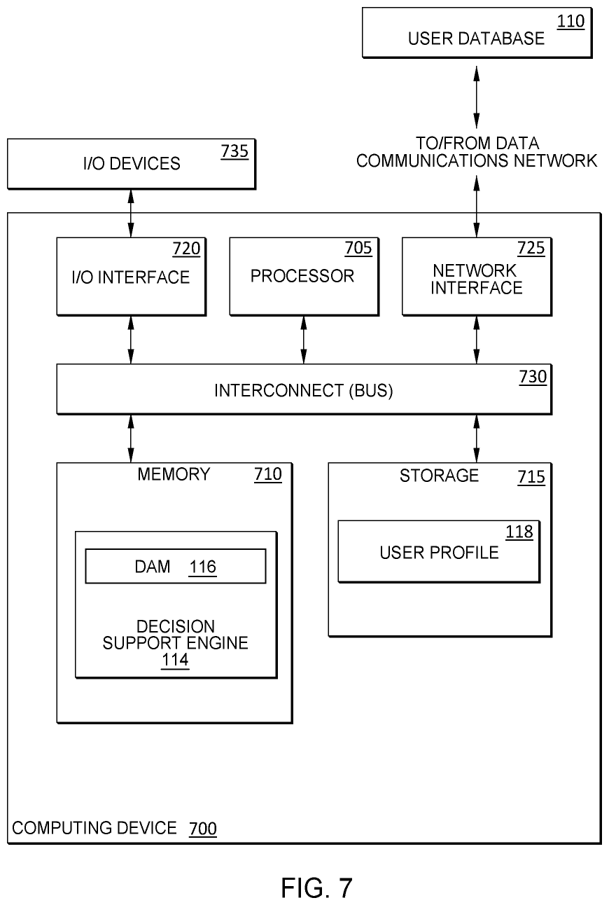

FIG. 7 is a block diagram depicting a computing device 700 configured to execute a decision support engine (e.g., decision support engine 114), according to certain embodiments disclosed herein. Although depicted as a single physical device, in embodiments, computing device 700 may be implemented using virtual device(s), and/or across a number of devices, such as in a cloud environment. As illustrated, computing device 700 includes a processor 705, memory 710, storage 715, a network interface 725, and one or more I/O interfaces 720. In the illustrated embodiment, processor 705 retrieves and executes programming instructions stored in memory 710, as well as stores and retrieves application data residing in storage 715. Processor 705 is generally representative of a single CPU and/or GPU, multiple CPUs and/or GPUs, a single CPU and/or GPU having multiple processing cores, and the like. Memory 710 is generally included to be representative of a random access memory. Storage 715 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, input and output (I/O) devices 735 (such as keyboards, monitors, etc.) can be connected via the I/O interface(s) 720. Further, via network interface 725, computing device 700 can be communicatively coupled with one or more other devices and components, such as user database 710. In certain embodiments, computing device 700 is communicatively coupled with other devices via a network, which may include the Internet, local network(s), and the like. The network may include wired connections, wireless connections, or a combination of wired and wireless connections. As illustrated, processor 705, memory 710, storage 715, network interface(s) 725, and I/O interface(s) 720 are communicatively coupled by one or more interconnects 730. In certain embodiments, computing device 700 is representative of mobile device 107 associated with the user. In certain embodiments, as discussed above, the mobile device 107 can include the user's laptop, computer, smartphone, and the like. In another embodiment, computing device 700 is a server executing in a cloud environment.

In the illustrated embodiment, storage 715 includes user profile 118. Memory 710 includes decision support engine 114, which itself includes DAM 116. Decision support engine 114 is executed by computing device 700 to perform operations 402-416 of method 400 in FIG. 4.

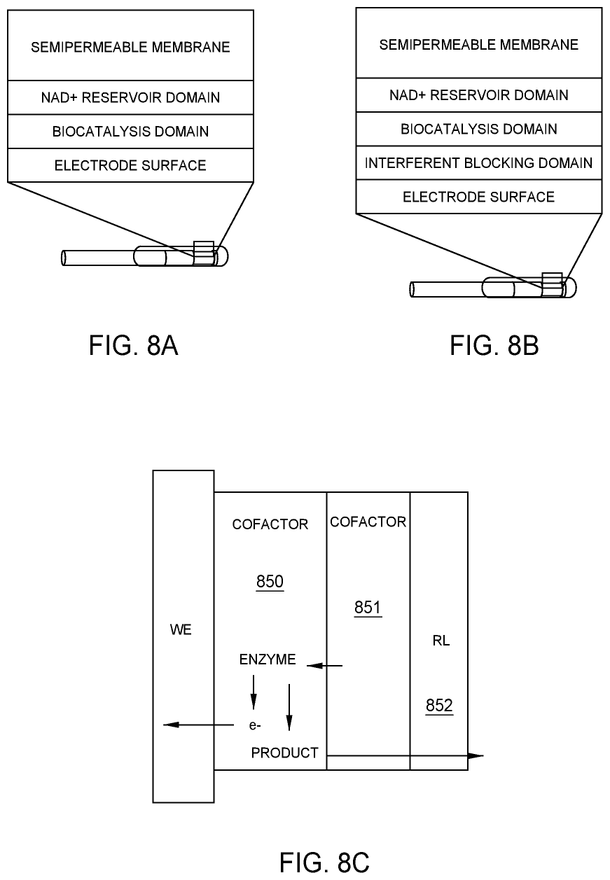

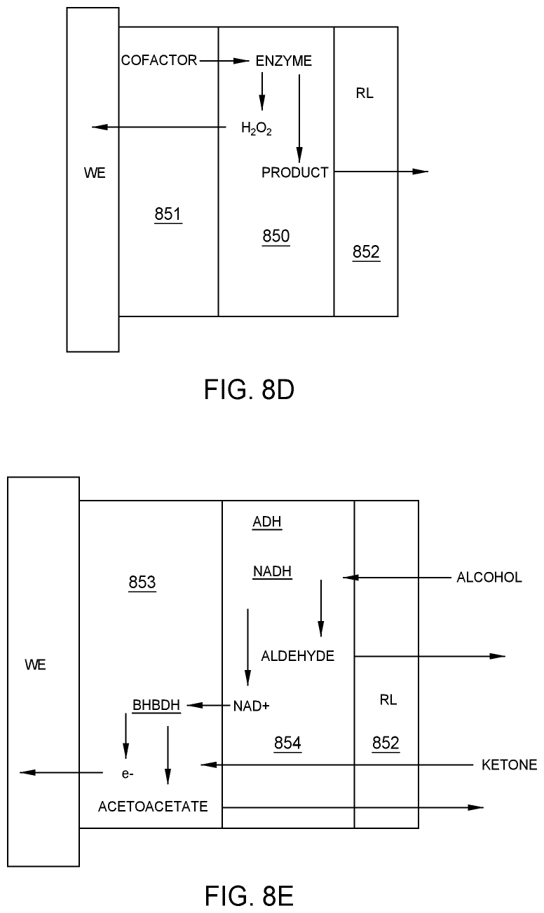

As described above, continuous analyte monitoring system 104, described in relation to FIG. 1, may be a multi-analyte sensor system including a multi-analyte sensor. FIG. 8A-12 describe example multi-analyte sensors used to measure multiple analytes.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include current and/or voltage, inductance, capacitance, impedance, transconductance, and potential. Optical properties include absorbance, fluorescence/phosphorescence, fluorescence/phosphorescence decay rate, wavelength shift, dual wave phase modulation, bio/chemiluminescence, reflectance, light scattering, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more DNA or RNA moieties. Transcutaneous continuous multi-analyte sensors can be used in vivo over various lengths of time. The continuous multi-analyte sensor systems discussed herein can be transcutaneous devices, in that a portion of the device may be inserted through the host's skin and into the underlying soft tissue while a portion of the device remains on the surface of the host's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the host's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps may be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (μm), or less, to about 1, 2, 3, 4, 5, 6, 7, 8, 9, 10, 11, 12, 13, 14, 15, or 16 μm is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl)carbodiimide (EDC)) and cured at a moderate temperature of about 50° C.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro)chemical reactions involved in sensing, but forms chemical bonds with the (electro)chemically active components of the membrane. In some examples these bonds are covalent. In one example, a tether may be formed in solution prior to one or more interlayers of a membrane being formed, where the tether bonds two (electro)chemically active components directly to one another or alternately, the tether(s) bond (electro)chemically active component(s) to polymeric backbone structures. In another example, (electro)chemically active components are comixed along with crosslinker(s) with tunable lengths (and optionally polymers) and the tethering reaction occurs as in situ crosslinking. Tethering may be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEGDE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip coating, vapor depositing, molding, 3-D printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor may promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein may include a biointerface layer. The biointerface layer, like the drug releasing layer, may include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein may include a drug releasing membrane at least partially functioning as or in combination with a biointerface membrane. The drug releasing membrane may include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

### Exemplary Multi Analyte Sensor Membrane Configurations

Continuous multi-analyte sensors with various membrane configurations suitable for facilitating signal transduction corresponding to analyte concentrations, either simultaneously, intermittently, and/or sequentially are provided. In one example, such sensors can be configured using a signal transducer, comprising one or more transducing elements (“TL”). Such continuous multi-analyte sensor can employ various transducing means, for example, amperometry, voltametric, potentiometry, and impedimetric methods, among other techniques.

In one example, the transducing element comprises one or more membranes that can comprise one or more layers and or domains, each of the one or more layers or domains can independently comprise one or more signal transducers, e.g., enzymes, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain may comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte-substrate/enzyme pairs: for example, sarcosine oxidase in combination with creatinine amidohydrolase, creatine amidohydrolase being employed for the sensing of creatinine. Other examples of analytes/oxidase enzyme combinations that can be used in the sensing region include, for example, alcohol/alcohol oxidase, cholesterol/cholesterol oxidase, glactose:galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine:xanthine/xanthine oxidase, glucose/glucose oxidase, lactate/lactate oxidase, L-amino acid oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, dimerized and/or fusion enzymes.

### NAD Based Multi Analyte Sensor Platform

Nicotinamide adenine dinucleotide (NAD(P)⁺/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)+) and reduced form (NAD(P)H) (H=hydrogen). The reaction of NAD+ and NADH is reversible, thus, the coenzyme can continuously cycle between the NAD(P)⁺/and NAD(P)H forms essentially without being consumed.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 8A. With reference to FIG. 8B, one or more optional layers may be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 8A-8B, one or more of the membranes provides a NAD+ reservoir domain providing a reservoir for NAD+. In one example, one or more interferent blocking membranes is used, and potentiostat is utilized to measure H₂O₂ production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein.

In one example, one or more mediators that are optimal for NADH oxidation are incorporated in the one or more electrode domains or enzyme domains. In one example, organic mediators, such as phenanthroline dione, or nitrosoanilines are used. In another example, metallo-organic mediators, such as ruthenium-phenanthroline-dione or osmium(bpy)₂Cl, polymers containing covalently coupled organic mediators or organometallic coordinated mediators polymers for example polyvinylimidizole-Os(bpy)₂Cl, or polyvinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a user or health care provider.

Another example of a continuous ketone analyte detection configuration employing electrode-associated mediator-coupled diaphorase/NAD+/dehydrogenase is depicted below:

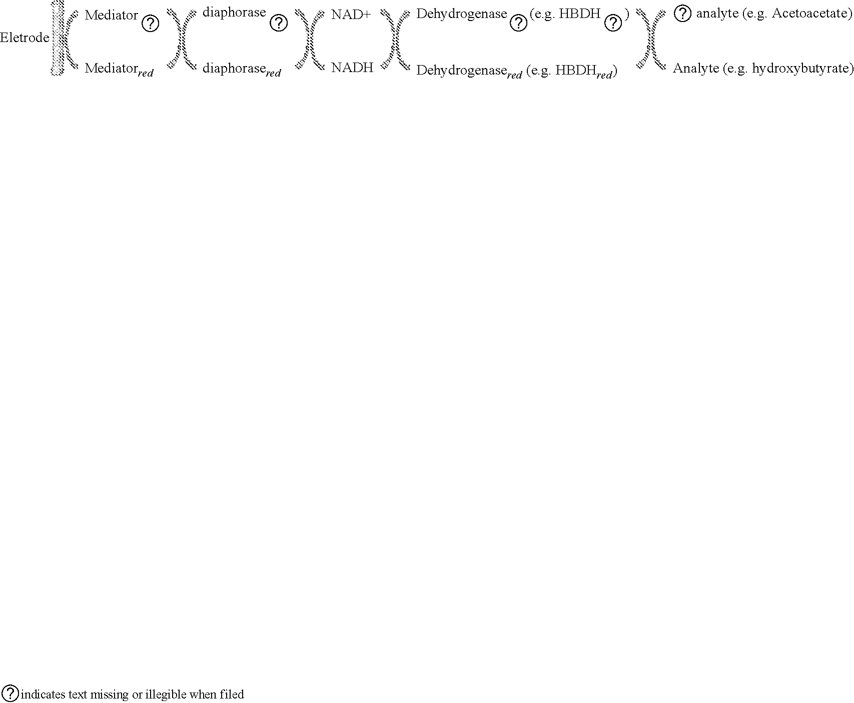

In one example, the diaphorase is electrically coupled to the electrode with organometallic coordinated mediator polymer. In another example, the diaphorase is covalently coupled to the electrode with an organometallic coordinated mediator polymer. Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte (ketone) oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ may be covalently coupled to an aspect of the enzyme domain having a higher molecular weight than the NAD+ which may improve a stability profile of the NAD+, improving the ability to retain and/or immobilize the NAD+ in the enzyme domain. For example, dextran-NAD.

In one example, the sensing region comprises one or more NADH:acceptor oxidoreductases and one or more NAD-dependent dehydrogenases. In one example, sensing region comprises one or more NADH:acceptor oxidoreductases and one or more NAD(P)-dependent dehydrogenases with NAD(P)+ or NAD(P)H as cofactors present in sensing region. In one example, the sensing region comprises an amount of diaphorase.

In one example, a ketone sensing configuration suitable for combination with another analyte sensing configuration is provided. Thus, an EZL layer of about 1-20 um thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400 MW], about 20 uL 500 mg/mL diaphorase, about 40 uL 250 mg/mL poly vinyl imidazole-osmium bis(2,2′-bipyridine)chloride (PVI-Os(bpy)₂Cl) to a substrate such as a working electrode, so as to provide, after drying, about 15-40% by weight HBDH, about 5-30% diaphorase about 5-30% NAD(P)H, about 10-50% PVI-Os(bpy)₂Cl and about 1-12% PEG-DGE (400 MW). The substrates discussed herein that may include working electrodes may be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% PVP, and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous ketone sensor as depicted in FIGS. 8A-8B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) may also contain a polymer or protein binder, such as zwitterionic polyurethane, and/or albumin. Alternatively, in addition to NAD(P)H, the membrane may contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer(or protein), and may be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE.

The aforementioned continuous ketone sensor configurations can be adapted to other analytes or used in combination with other sensor configurations. For example, analyte(s)-dehydrogenase enzyme combinations can be used in any of the membranes of the sensing region include; glycerol (glycerol dehydrogenase); cortisol (11β-hydroxysteroid dehydrogenase); glucose (glucose dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. FIG. 1C depicts this exemplary configuration, of an enzyme domain 850 comprising an enzyme (Enzyme) with an amount of cofactor (Cofactor) that is positioned proximal to at least a portion of a working electrode (“WE”) surface, where the WE comprises an electrochemically reactive surface. In one example, a second membrane 851 comprising an amount of cofactor is positioned adjacent the first enzyme domain. The amount of cofactor in the second membrane can provide an excess for the enzyme, e.g., to extend sensor life. One or more resistance domains 852 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 8D depicts an alternative enzyme domain configuration comprising a first membrane 851 with an amount of cofactor that is positioned more proximal to at least a portion of a WE surface. Enzyme domain 850 comprising an amount of enzyme is positioned adjacent the first membrane.

In the membrane configurations depicted in FIGS. 8C-8D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

FIG. 8E depicts another continuous multi-analyte membrane configuration, where {beta}-hydroxybutyrate dehydrogenase BHBDH in a first enzyme domain 853 is positioned proximate to a working electrode WE and second enzyme domain 1854, for example, comprising alcohol dehydrogenase (ADH) and NADH is positioned adjacent the first enzyme domain. One or more resistance domains RL 852 may be deployed adjacent to the second enzyme domain 854. In this configuration, the presence of the combination of alcohol and ketone in serum works collectively to provide a transduced signal corresponding to at least one of the analyte concentrations, for example, ketone. Thus, as the NADH present in the more distal second enzyme domain consumes alcohol present in the serum environment, NADH is oxidized to NAD(P)H that diffuses into the first membrane layer to provide electron transfer of the BHBDH catalysis of acetoacetate ketone and transduction of a detectable signal corresponding to the concentration of the ketone. In one example, an enzyme can be configured for reverse catalysis and can create a substrate used for catalysis of another enzyme present, either in the same or different layer or domain. Other configurations can be used in the aforementioned configuration, such as electrode, resistance, bio-interfacing, and drug releasing membranes, layers, or domains. Thus, a first enzyme domain that is more distal from the WE than a second enzyme domain may be configured to generate a cofactor or other element to act as a reactant (and/or a reactant substrate) for the second enzyme domain to detect the one or more target analytes.

### Alcohol Sensor Configurations

In one example, a continuous alcohol (e.g., ethanol) sensor device configuration is provided. In one example, one or more enzyme domains comprising alcohol oxidase (AOX) is provided and the presence and/or amount of alcohol is transduced by creation of hydrogen peroxide, alone or in combination with oxygen consumption or with another substrate-oxidase enzyme system, e.g., glucose-glucose oxidase, in which hydrogen peroxide and or oxygen and/or glucose can be detected and/or measured qualitatively or quantitatively, using amperometry.

In one example, the sensing region for the aforementioned enzyme substrate-oxidase enzyme configurations has one or more enzyme domains comprises one or more electrodes. In one example, the sensing region for the aforementioned enzyme substrate-oxidase enzyme configurations has one or more enzyme domains, with or without the one or more electrodes, further comprises one or interference blocking membranes (e.g. permselective membranes, charge exclusion membranes) to attenuate one or more interferents from diffusing through the membrane to the working electrode. In one example, the sensing region for the aforementioned substrate-oxidase enzyme configurations has one or more enzyme domains, with or without the one or more electrodes, and further comprises one or resistance domains with or without the one or more interference blocking membranes to attenuate one or more analytes or enzyme substrates. In one example, the sensing region for the aforementioned substrate-oxidase enzyme configurations has one or more enzyme domains, with or without the one or more electrodes, one or more resistance domains with or without the one or more interference blocking membranes further comprises one or biointerface membranes and/or drug releasing membranes, independently, to attenuate one or more analytes or enzyme substrates and attenuate the immune response of the host after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate alcohol itself as well as attenuate other electrochemically actives species or other analytes that can otherwise interfere by producing a signal if they diffuse to the working electrode.

In one example, the working electrode used comprised platinum and the potential applied was about 0.5 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of alcohol. When appropriately designed to obey stoichiometric behavior, the presence of a specific concentration of alcohol should cause a commensurate reduction in local oxygen in a direct (linear) relation with the concentration of alcohol. Accordingly, a multi-analyte sensor for both alcohol and oxygen can therefore be provided.

In another example, the above mentioned alcohol sensing configuration can include one or more secondary enzymes that react with a reaction product of the alcohol/alcohol oxidase catalysis, e.g., hydrogen peroxide, and provide for a oxidized form of the secondary enzyme that transduces an alcohol-dependent signal to the WE/RE at a lower potential than without the secondary enzyme. Thus, in one example, the alcohol/alcohol oxidase is used with a reduced form of a peroxidase, for example horse radish peroxidase. The alcohol/alcohol oxidase can be in same or different layer as the peroxidase, or they may be spatially separated distally from the electrode surface, for example, the alcohol/alcohol oxidase being more distal from the electrode surface and the peroxidase being more proximal to the electrode surface, or alternatively, the alcohol/alcohol oxidase being more proximal from the electrode surface and the peroxidase being more distal to the electrode surface. In one example, the alcohol/alcohol oxidase, being more distal from the electrode surface and the peroxidase, further includes any combination of electrode, interference, resistance, and biointerface membranes to optimize signal, durability, reduce drift, or extend end of use duration.

In another example, the above mentioned alcohol sensing configuration can include one or more mediators. In one example, the one or more mediators are present in, on, or about one or more electrodes or electrode surfaces and/or are deposited or otherwise associated with the surface of the working electrode (WE) or reference electrode (RE). In one example, the one or more mediators eliminate or reduce direct oxidation of interfering species that may reach the WE or RE. In one example, the one or more mediators provide a lowering of the operating potential of the WE/RE, for example, from about 0.6V to about 0.3V or less on a platinum electrode, which can reduce or eliminates oxidation of endogenous interfering species. Examples of one or mediators are provided below. Other electrodes, e.g., counter electrodes, can be employed.

In one example, other enzymes or additional components may be added to the polymer mixture(s) that constitute any part of the sensing region to increase the stability of the aforementioned sensor and/or reduce or eliminate the biproducts of the alcohol/alcohol oxidase reaction. Increasing stability includes storage or shelf life and/or operational stability (e.g., retention of enzyme activity during use). For example, byproducts of enzyme reactions may be undesirable for increased shelf life and/or operational stability, and may thus be desirable to reduce or remove. In one example, xanthine oxidase can be used to remove bi-products of one or more enzyme reactions.

In another example, a dehydrogenase enzyme is used with a oxidase for the detection of alcohol alone or in combination with oxygen. Thus, in one example, alcohol dehydrogenase is used to oxidize alcohol to aldehyde in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled (e.g., “wired”) alcohol dehydrogenase (ADH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+ may be coupled to a polymer, such as dextran, the polymer immobilized in the enzyme domain along with ADH. This provides for retention of the co-factor and availability thereof for the active site of ADH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface is provided. A chemical moiety capable of assisting with electron transfer from the enzyme or cofactor to the electrode surface can be used and includes one or more mediators as described below.

In one example, any one of the aforementioned continuous alcohol sensor configurations are combined with any one of the aforementioned continuous ketone monitoring configurations to provide a continuous multi-analyte sensor device as further described below. In one example a continuous glucose monitoring configuration combined with any one of the aforementioned continuous alcohol sensor configurations and any one of the aforementioned continuous ketone monitoring configurations to provide a continuous multi-analyte sensor device as further described below.

### Uric Acid Sensor Configurations

In another example, a continuous uric acid sensor device configuration is provided. Thus, in one example, uric acid oxidase (UOX) can be included in one or more enzyme domains and positioned adjacent the working electrode surface. The catalysis of the uric acid using UOX, produces hydrogen peroxide which can be detected using, among other techniques, amperometry, voltametric and impedimetric methods. In one example, to reduce or eliminate the interference from direct oxidation of uric acid on the electrode surface, one or more electrode, interference, and/or resistance domains can be deposited on at least a portion of the working electrode surface. Such membranes can be used to attenuate diffusion of uric acid as well as other analytes to the working electrode that can interfere with signal transduction.

In one alternative example, a uric acid continuous sensing device configuration comprises sensing oxygen level changes about the WE surface, e.g., for example, as in a Clark type electrode setup, or the one or more electrodes can comprise, independently, one or more different polymers such as NAFION™, polyzwitterion polymers, or polymeric mediator adjacent at least a portion of the electrode surface. In one example, the electrode surface with the one or more electrode domains provide for operation at a different or lower voltage to measure oxygen. Oxygen level and its changes in can be sensed, recorded, and correlated to the concentration of uric acid based using, for example, using conventional calibration methods.

In one example, alone or in combination with any of the aforementioned configurations, uric acid sensor configurations, so as to lower the potential at the WE for signal transduction of uric acid, one or more coatings can be deposited on the WE surface. The one or more coatings may be deposited or otherwise formed on the WE surface and/or on other coatings formed thereon using various techniques including, but not limited to, dipping, electrodepositing, vapor deposition, spray coating, etc. In one example, the coated WE surface can provide for redox reactions, e.g., of hydrogen peroxide, at lower potentials (as compared to 0.6 V on platinum electrode surface without such a coating. Example of materials that can be coated or annealed onto the WE surface includes, but are not limited to Prussian Blue, Medola Blue, methylene blue, methylene green, methyl viologen, ferrocyanide, ferrocene, cobalt ion, and cobalt phthalocyanine, and the like.

In one example, one or more secondary enzymes, cofactors and/or mediators (electrically coupled or polymeric mediators) can be added to the enzyme domain with UOX to facilitate direct or indirect electron transfer to the WE. In such configurations, for example, regeneration of the initial oxidized form of secondary enzyme is reduced by the WE for signal transduction. In one example, the secondary enzyme is horse radish peroxidase (HRP).

### Choline Sensor Configurations

In one example continuous choline sensor device can be provided, for example, using choline oxidase enzyme that generates hydrogen peroxide with the oxidation of choline. Thus, in one example, at least one enzyme domain comprises choline oxidase (COX) adjacent at least one WE surface, optionally with one or more electrodes and/or interference membranes positioned in between the WE surface and the at least one enzyme domain. The catalysis of the choline using COX results in creation of hydrogen peroxide which can be detectable using, among other techniques, amperometry, voltametric and impedimetric methods.

In one example, the aforementioned continuous choline sensor configuration is combined with any one of the aforementioned continuous alcohol sensor configurations, and continuous uric acid sensor configurations to provide a continuous multi-analyte sensor device as further described below. This continuous multi-analyte sensor device can further include continuous glucose monitoring capability. Other membranes can be used in the aforementioned continuous choline sensor configuration, such as electrode, resistance, bio-interfacing, and drug releasing membranes.

### Cholesterol Sensor Configurations

In one example, continuous cholesterol sensor configurations can be made using cholesterol oxidase (CHOX), in a manner similar to previously described sensors. Thus, one or more enzyme domains comprising CHOX can be positioned adjacent at least one WE surface. The catalysis of free cholesterol using CHOX results in creation of hydrogen peroxide which can be detectable using, among other techniques, amperometry, voltametric and impedimetric methods.

An exemplary cholesterol sensor configuration using a platinum WE, where at least one interference membrane is positioned adjacent at least one WE surface, over which there is at least one enzyme domain comprising CHOX, over which is positioned at least one resistance domain to control diffusional characteristics was prepared.

The method described above and the cholesterol sensors described can measure free cholesterol, however, with modification, the configuration can measure more types of cholesterol as well as total cholesterol concentration. Measuring different types of cholesterol and total cholesterol is important, since due to low solubility of cholesterol in water significant amount of cholesterol is in unmodified and esterified forms. Thus, in one example, a total cholesterol sample is provided where a secondary enzyme is introduced into the at least one enzyme domain, for example, to provide the combination of cholesterol esterase with CHOX Cholesteryl ester, which essentially represents total cholesterols can be measured indirectly from signals transduced from cholesterol present and formed by the esterase.

In one example, the aforementioned continuous (total) cholesterol sensor configuration is combined with any one of the aforementioned continuous alcohol sensor configurations and/or continuous uric acid sensor configurations to provide a continuous multi-analyte sensor system as further described below. This continuous multi-analyte sensor device can further include continuous glucose monitoring capability. Other membrane configurations can be used in the aforementioned continuous cholesterol sensor configuration, such as one or more electrode domains, resistance domains, bio-interfacing domains, and drug releasing membranes.

### Bilirubin Sensor and Ascorbic Acid Sensor Configurations

In one example, continuous bilirubin and ascorbic acid sensors are provided. These sensors can employ bilirubin oxidase and ascorbate oxidase, respectively. However, unlike some oxidoreductase enzymes, the final product of the catalysis of analytes of bilirubin oxidase and ascorbate oxidase is water instead of hydrogen peroxide. Therefore, redox detection of hydrogen peroxide to correlate with bilirubin or ascorbic acid is not possible. However, these oxidase enzymes still consume oxygen for the catalysis, and the levels of oxygen consumption correlates with the levels of the target analyte present. Thus, bilirubin and ascorbic acid levels can be measured indirectly by electrochemically sensing oxygen level changes, as in a Clark type electrode setup, for example.

Alternatively, a different configuration for sensing bilirubin and ascorbic acid can be employed. For example, an electrode domain including one or more electrode domains comprising electron transfer agents, such as NAFION™, polyzwitterion polymers, or polymeric mediator can be coated on the electrode. Measured oxygen levels transduced from such enzyme domain configurations can be correlated with the concentrations of bilirubin and ascorbic acid levels. In one example, an electrode domain comprising one or more mediators electrically coupled to a working electrode can be employed and correlated to the levels of bilirubin and ascorbic acid levels.

In one example, the aforementioned continuous bilirubin and ascorbic acid sensor configurations can be combined with any one of the aforementioned continuous alcohol sensor configurations, continuous uric acid sensor configurations, continuous cholesterol sensor configurations to provide a continuous multi-analyte sensor device as further described below. This continuous multi-analyte sensor device can further include continuous glucose monitoring capability. Other membranes can be used in the aforementioned continuous bilirubin and ascorbic acid sensor configuration, such as electrode, resistance, bio-interfacing, and drug releasing membranes.

### One-Working-Electrode Configurations for Dual Analyte Detection

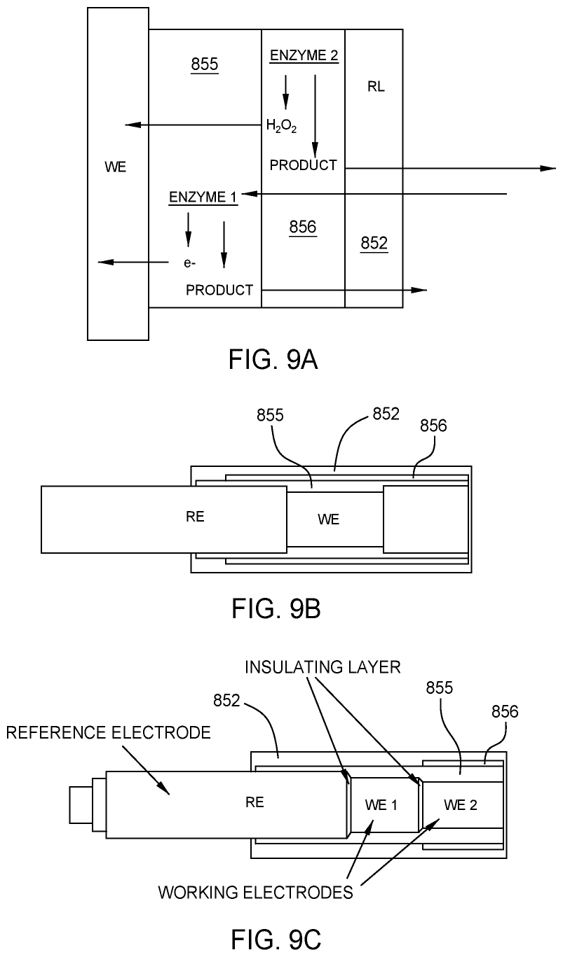

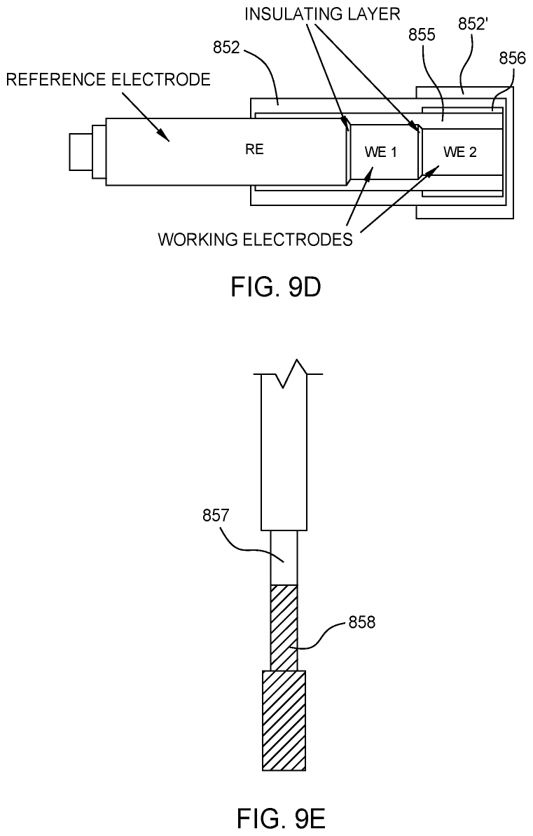

In one example, at least a dual enzyme domain configuration in which each layer contains one or more specific enzymes and optionally one or more cofactors is provided. In a broad sense, one example of a continuous multi-analyte sensor configuration is depicted in FIG. 9A where a first membrane 855 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 856 (EZL2) with at least one second enzyme (Enzyme 2) is positioned adjacent 855 ELZ1, and is generally more distal from WE than EZL1. One or more resistance domains (RL) 852 can be provided adjacent EZL2 856, and/or between EZL1 855 and EZL2 856. The different enzymes catalyze the transformation of the same analyte, but at least one enzyme in EZL2 856 provides hydrogen peroxide and the other at least one enzyme in EZL1 855 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 9A, a first analyte diffuses through RL 852 and into EZL2 856 resulting in peroxide via interaction with Enzyme 2. Peroxide diffuses at least through EZL1 855 to WE and transduces a signal that corresponds directly or indirectly to the first analyte concentration. A second analyte, which is different from the first analyte, diffuses through RL 852 and EZL2 856 and interacts with Enzyme 1, which results in electron transfer to WE and transduces a signal that corresponds directly or indirectly to the second analyte concentration.

As shown in FIG. 9B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 856 providing hydrogen peroxide and the at least other enzyme in EZL1 855 not providing hydrogen peroxide, e.g., providing electron transfer to the WE surface corresponding directly or indirectly to a concentration of the analyte.

In one example, an inner layer of the at least two enzyme domains EZL1, EZL2 855, 856 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 855 is more proximal to the WE surface and may have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 855 is directly adjacent the WE.

The second layer of at least dual enzyme domain (the outer layer EZL2 856) of FIG. 9B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 856 and through the inner layer EZL1 855 to reach the WE surface and undergoes redox at a potential of P2, where P2 P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry etc. In some examples, impedimetric sensing may be used. In one example, a phase shift (e.g., a time lag) may result from detecting two signals from two different working electrodes, each signal being generated by a different EZL (EZL1, EZL2, 855, 856) associated with each electrode. The two (or more) signals can be broken down into components to detect the individual signal and signal artifacts generated by each of EZL1 855 and EZL2 856 in response to the detection of two analytes. In some examples, each EZL detects a different analyte. In other examples, both EZLs detect the same analyte.

In another alternative exemplary configuration, as shown in FIGS. 9C-9D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2),In one example, the first analyte detected by WE1 is glucose, and the second analyte detected by WE2 is lactate. In another example, the first analyte detected by WE1 is glucose, and the second analyte detected by WE2 is ketones.

Thus, FIGS. 9C-9D depict exemplary configurations of a continuous multi-analyte sensor construct in which EZL1 855, EZL2 856 and RL 852 (resistance domain) as described above, arranged, for example, by sequential dip coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 9C-9D, WE1 represents a first working electrode surface configured to operate at P1, for example, and is electrically insulated from second working electrode surface WE2 that is configured to operate at P2, and RE represents a reference electrode RE electrically isolated from both WE1, WE2. One resistance domain is provided in the configuration of FIG. 9C that covers the reference electrode and WE1, WE2. An addition resistance domain is provided in the configuration of FIG. 9D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (H₂O₂ producing) and glucose dehydrogenase (electrically coupled) configuration. Measurement of Glucose at two potentials and from two different electrodes provides more data points and accuracy. Such approaches may not be needed for glucose sensing, but the can be applied across the biomarker sensing spectrum of other analytes, alone or in combination with glucoses sensing, such as ketone sensing, ketone/lactate sensing, and ketone/glucose sensing.

In an alternative configuration of that depicted in FIGS. 9C-9D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 9D, such an arrangement of RL's is depicted, where an additional RL 852′ is adjacent WES2 but substantially absent from WES1.

In one example of measuring two different analytes, the above configuration comprising enzyme domain EZL1 855 comprising one or more enzyme(s) and one or more mediators for at least one enzyme of EZL1 to provide for direct electron transfer to the WES1 and determining a concentration of at least a first analyte. In addition, enzyme domain EZL2 856 can comprise at least one enzyme that provides peroxide (e.g., hydrogen peroxide) or consumes oxygen during catalysis with its substrate. The peroxide or the oxygen produced in EZL2 856 migrates to WES2 and provides a detectable signal that corresponds directly or indirectly to a second analyte. For example, WES2 can be carbon, wired to glucose dehydrogenase to measure glucose, while WES1 can be platinum, that measures peroxided produced from lactate oxidase/lactate in EZL2 856. The combinations of electrode material and enzyme(s) as disclosed herein are examples and non-limiting.

In one example, the potentials of P1 and P2 can be separated by an amount of potential so that both signals (from direct electron transfer from EZL1 855 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non-limiting and can include different amperometry techniques, e.g., cyclic voltammetry. In one example, an alternative configuration is provided but hydrogen peroxide production in EZL2 is replaced by another suitable electrolysis compound that maintains the P2 P1 relationship, such as oxygen, and at least one enzyme-substrate combination that provide the other electrolysis compound.

For example, a continuous multi-analyte sensor configuration, for choline and glucose, in which enzyme domains EZ1 855, EZ2 856 were associated with different WEs, e.g., platinum WE2, and gold WE1 was prepared. In this exemplary case, EZL1 855 contained glucose oxidase and a mediator coupled to WE1 to facilitate electron direct transfer upon catalysis of glucose, and EZL2 856 contained choline oxidase that will catalyze choline and generate hydrogen peroxide for electrolysis at WE2. The EZL's were coated with resistance domains; upon cure and readiness they underwent cyclic voltammetry in the presence of glucose and choline. A wired glucose oxidase enzyme to a gold electrode is capable of transducing signal at 0.2 volts, therefore, by analyzing the current changes at 0.2 volts, the concentration of glucose can be determined. The data also demonstrates that choline concentration is also inferentially detectable at the WE2 platinum electrode if the CV trace is analyzed at the voltage P2.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 9E, an example of such composite electrode surfaces is shown, in which an extended platinum covered wire 857 is half coated with carbon 858, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that may be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include ketone sensing (beta-hydroxybutyrate dehydrogenase electrically coupled enzyme in EZL1 855) and glucose sensing (glucose oxidase in EZL2 856). Other membranes can be used in the aforementioned configuration, such as electrode, resistance, bio-interfacing, and drug releasing membranes. In other examples, one or both of the working electrodes (WE1, WE2) may be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein may instead or additionally include graphene, graphene oxide, or other materials suitable for forming the working electrodes, such as commercially available carbon ink.

### Glycerol Sensor Configurations

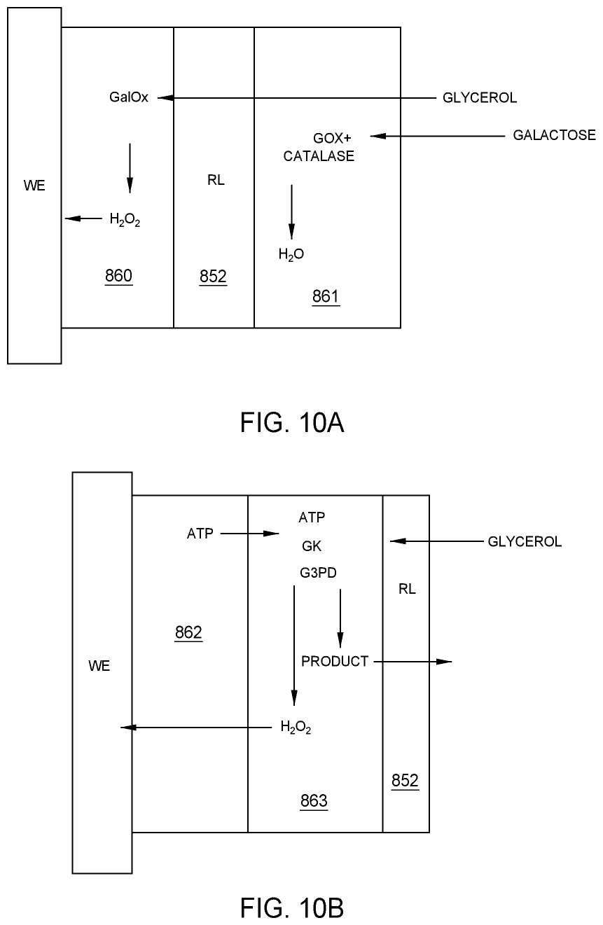

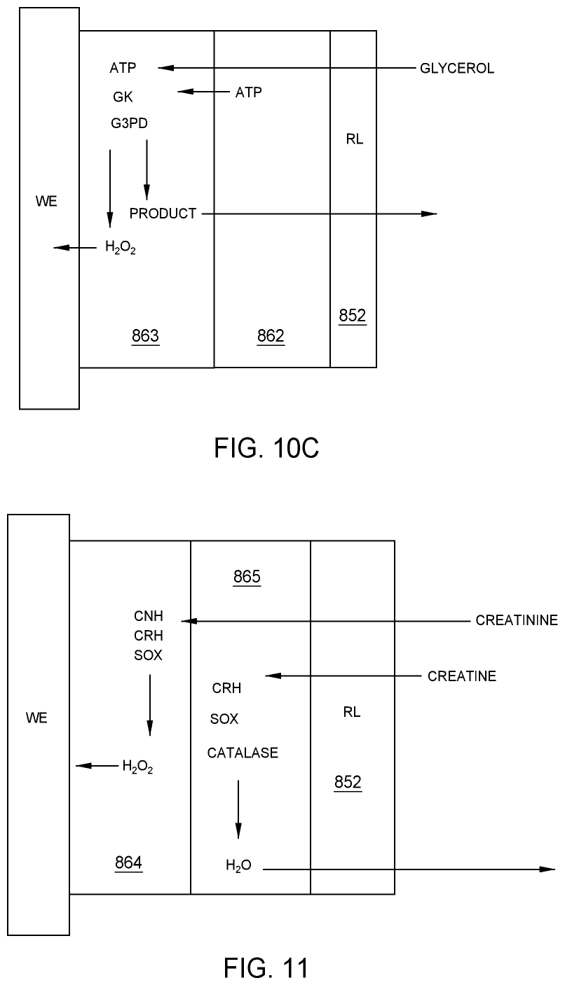

As shown in FIG. 10A, an exemplary continuous glycerol sensor configuration is depicted where a first enzyme domain EZL1 860 comprising galactose oxidase is positioned proximal to at least a portion of a WE surface. A second enzyme domain EZL2 861 comprising glucose oxidase and catalase is positioned more distal from the WE. As shown in FIG. 10A, one or more resistance domains (RL) 852 are positioned between EZL1 860 and EZL2 861. Additional RLs can be employed, for example, adjacent to EZL2 861. Modification of the one or more RL membranes to attenuate the flux of either analyte and increase glycerol to galactose sensitivity ratio is envisaged. The above glycerol sensing configuration provides for a glycerol sensor that can be combined with one or more additional sensor configurations as disclosed herein.

Glycerol can be catalyzed by the enzyme galactose oxidase (GalOx), however, GalOx has an activity ratio of 1%-5% towards glycerol. In one example, the activity of GalOx towards this secondary analyte glycerol can be utilized. The relative concentrations of glycerol in vivo are much higher that galactose (˜2 umol/l for galactose, and ˜100 umol/l for glycerol), which compliments the aforementioned configurations.

If the GalOx present in EZL1 860 membrane is not otherwise functionally limited, then the GalOx will catalyze most if not all of the glycerol that passes through the one or more RLs. The signal contribution from the glycerol present will be higher as compared to the signal contribution from galactose. In one example, the one or more RL's are chemically configured to provide a higher influx of glycerol or a lower influx of galactose.

In another example, a glycol sensor configuration is provided using multiple working electrodes WEs that provides for utilizing signal transduced from both WEs. Utilizing signal transduced from both WEs can provide increasing selectivity. In one example EZL1 860 and EZL2 861 comprise the same oxidase enzyme (e.g., galactose oxidase) with different ratios of enzyme loading, and/or a different immobilizing polymer and/or different number and layers of RL's over the WEs. Such configurations provide for measurement of the same target analyte with different sensitivities, resulting in a dual measurement. Using a mathematical algorithm to correct for noise and interference from a first signal, and inputting the first signal from one sensing electrode with a first analyte sensitivity ratio into the mathematical algorithm, allows for the decoupling of the second signal corresponding to the desired analyte contributions. Modification of the sensitivity ratio of the one or more EZL's to distinguish signals from the interfering species and the analyte(s) of interest can be provided by adjusting one or more of enzyme source, enzyme load in EZL's, chemical nature/diffusional characteristics of EZL's, chemical/diffusional characteristics of the at least one RL's, and combinations thereof.

As discussed herein, a secondary enzyme domain can be utilized to catalyze the non-target analyte(s), reducing their concentration and limiting diffusion towards the sensing electrode through adjacent membranes that contains the primary enzyme and necessary additives. In this example, the most distal enzyme domain, EZL2, 861 is configured to catalyze a non-target analyte that would otherwise react with EZL1, thus providing a potentially less accurate reading of the target analyte (glycerol) concentration. This secondary enzyme domain can act as a “selective diffusion exclusion membrane” by itself, or in some other configurations can be placed above or under a resistant layer (RL) 852. In this example, the target analyte is glycerol and GalOX is used to catalyze glycerol to form a measurable species (e.g., hydrogen peroxide).

In one example, a continuous glycerol sensor configuration is provided using at least glycerol oxidase, which provides hydrogen peroxide upon reaction and catalysis of glycerol. Thus, in one example, enzyme domain comprising glycerol oxidase can be positioned adjacent at least a portion of a WE surface and hydrogen peroxide is detected using amperometry. In another example, enzyme domain comprising glycerol oxidase is used for sensing oxygen level changes, for example, in a Clark type electrode setup. Alternatively, at least a portion of the WE surface can be coated with one more layers of electrically coupled polymers, such as a mediator system discussed below, to provide a coated WE capable of electron transfer from the enzyme at a lower potential. The coated WE can then operate at a different and lower voltage to measure oxygen and its correlation to glycerol concentration.

In another example, a glycerol sensor configuration is provided using glycerol-3-phosphate oxidase in the enzyme domain. In one example, ATP is used as the cofactor. Thus, as shown in FIGS. 10B and 10C, exemplary sensor configurations are depicted where in one example (FIG. 10B), one or more cofactors (e.g. ATP) 862 is proximal to at least a portion of an WE surface. One or more enzyme domains 863 comprising glycerol-3-phospohate oxidase (G3PD), lipase, and/or glycerol kinase (GK) and one or more regenerating enzymes capable of continuously regenerating the cofactor are contained in an enzyme domain are adjacent the cofactor, or more distal from the WE surface than the cofactor layer 862. Examples of regenerating enzymes that can be used to provide ATP regeneration include, but are not limited to, ATP synthase, pyruvate kinase, acetate kinase, and creatine kinase. The one or more regenerating enzymes can be included in one or more enzyme domains, or in a separate layer.

An alternative configuration is shown in FIG. 10C, where one or more enzyme domains 863 comprising G3PD, at least one cofactor and at least one regenerating enzyme, are positioned proximal to at least a portion of WE surface, with one or more cofactor reservoirs 862 adjacent to the enzyme domains comprising G3PD and more distal from the WE surface, and one or more RL's 852 are positioned adjacent the cofactor reservoir. In either of these configurations, an additional enzyme domain comprising lipase can be included to indirectly measure triglyceride, as the lipase will produce glycerol for detection by the aforementioned glycerol sensor configurations.

In another example, a glycerol sensor configuration is provided using dehydrogenase enzymes with cofactors and regenerating enzymes. In one example, cofactors that can be incorporated in the one or more enzyme domains include one or more of NAD(P)H, NADP+, and ATP. In one example, e.g., for use of NAD(P)H a regenerating enzyme can be NADH oxidase or diaphorase to convert NADH, the product of the dehydrogenase catalysis back to NAD(P)H. Similar methodologies can be used for creating other glycerol sensors, for example, glycerol dehydrogenase, combined with NADH oxidase or diaphorase can be configured to measure glycerol or oxygen.

In one example, mathematical modeling can be used to identify and remove interference signals, measuring very low analyte concentrations, signal error and noise reduction so as to improve and increase of multi-analyte sensor end of life. For example, with a two WE electrode configuration where WE1 is coated with a first EZL while WE2 is coated with two or more different EZL, optionally with one or more resistance domains (RL) a mathematical correction such interference can be corrected for, providing for increasing accuracy of the measurements.

Changes of enzyme load, immobilizing polymer and resistance domain characteristics over each analyte sensing region can result in different sensitive ratios between two or more target analyte and interfering species. If the signal are collected and analyzed using mathematical modeling, a more precise concentration of the target analytes can be calculated.

One example in which use of mathematical modeling can be helpful is with glycerol sensing, where galactose oxidase is sensitive towards both galactose and glycerol. The sensitivity ratio of galactose oxidase to glycerol is about is 1%-5% of its sensitivity to galactose. In such case, modification of the sensitivity ratio to the two analytes is possible by adjusting the one or more parameters, such as enzyme source, enzyme load, enzyme domain (EZL) diffusional characteristics, RL diffusional characteristics, and combinations thereof. If two WEs are operating in the sensor system, signal correction and analysis from both WEs using mathematical modeling provides high degree of fidelity and target analyte concentration measurement.

In the above configurations, the proximity to the WE of one or more of these enzyme immobilizing layers discussed herein can be different or reversed, for example if the most proximal to the WE enzyme domain provides hydrogen peroxide, this configuration can be used.

In some examples, the target analyte can be measured using one or multiple of enzyme working in concert. In one example, ATP can be immobilized in one or more EZL membranes, or can be added to an adjacent layer alone or in combination with a secondary cofactor, or can get regenerated/recycled for use in the same EZL or an adjacent third EZL. This configuration can further include a cofactor regenerator enzyme, e.g., alcohol dehydrogenase or NADH oxidase to regenerate NAD(P)H. Other examples of cofactor regenerator enzymes that can be used for ATP regeneration are ATP synthase, pyruvate kinase, acetate kinase, creatine kinase, and the like.

In one example, the aforementioned continuous glycerol sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations to provide a continuous multi-analyte sensor device as further described below. This continuous multi-analyte sensor device can further include continuous glucose monitoring capability. Other configurations can be used in the aforementioned continuous glycerol sensor configuration, such as electrode, resistance, bio-interfacing, and drug releasing membranes.

### Creatinine Sensor Configurations

In one example, continuous creatinine sensor configurations are provided, such configurations containing one or more enzymes and/or cofactors. Creatinine sensor configurations are examples of continuous analyte sensing systems that generate intermediate, interfering products, where these intermediates/interferents are also present in the biological fluids sampled. The present disclosure provides solutions to address these technical problems and provide for accurate, stable, and continuous creatinine monitoring alone or in combination with other continuous multi-analyte sensor configurations.

Creatinine sensors, when in use, are subject to changes of a number of physiologically present intermediate/interfering products, for example sarcosine and creatine, that can affect the correlation of the transduced signal with the creatinine concentration. The physiological concentration range of sarcosine, for example, is an order of magnitude lower that creatinine or creatine, so signal contribution from circulating sarcosine is typically minimal. However, changes in local physiological creatine concentration can affect the creatinine sensor signal. In one example, eliminating or reducing such signal contribution is provided.

Thus, in one example, eliminating or reducing creatine signal contribution of a creatinine sensor comprises using at least one enzyme that will consume the non-targeted interfering analyte, in this case, creatine. For example, two enzyme domains are used, positioned adjacent to each other. At least a portion of a first enzyme domain is positioned proximal to at least a portion of a WE surface, the first enzyme domain comprising one or more enzymes selected from creatinine amidohydrolase (CNH), creatine amidohydrolase (CRH), and sarcosine oxidase (SOX). A second enzyme domain, adjacent the first enzyme domain and more distal from the WE surface, comprises one or more enzymes using creatine as their substrate so as to eliminate or reduce creatine diffusion towards the WE. In one example, combinations of enzymes include CRH, SOX, creatine kinase, and catalase, where the enzyme ratios are tuned to provide ample number of units such that circulating creatine will at least partially be consumed by CRH providing sarcosine and urea, whereas the sarcosine produced will at least partially be consumed by SOX, providing an oxidized form of glycine (e.g. glycine aldehyde) which will at least be partially consumed by catalase. In an alternative configuration of the above, the urea produced by the CRH catalysis can at least partially be consumed by urease to provide ammonia, with the aqueous form (NH4+) being detected via an ion-selective electrode (e.g., nonactin ionophore). Such an alternative potentiometric sensing configuration may provide an alternative to amperometric peroxide detection (e.g., improved sensitivity, limits of detection, and lack of depletion of the reference electrode, alternate pathways/mechanisms). This dual-analyte-sensing example may include a creatinine-potassium sensor having potentiometric sensing at two different working electrodes. In this example, interference signals can be identified and corrected. In one alternative example, the aforementioned configuration can include multi-modal sensing architectures using a combination of amperometry and potentiometry to detect concentrations of peroxide and ammonium ion, measured using amperometry and potentiometry, respectively, and correlated to measure the concentration of the creatinine. In one example, the aforementioned configurations can further comprise one or more configurations (e.g., without enzyme) separating the two enzyme domains to provide complementary or assisting diffusional separations and barriers.

In yet another example, a method to isolate the signal and measure essentially only creatinine is to use a second WE that measures the interfering species (e.g., creatine) and then correct for the signal using mathematical modeling. Thus, for example, signal from the WE interacting with creatine is used as a reference signal. Signal from another WE interacting with creatinine is from corrected for signal from the WE interacting with creatine to selectively determine creatinine concentration.

In yet another example, sensing creatinine is provided by measuring oxygen level changes electrochemically, for example in a Clark type electrode setup, or using one or more electrodes coated with layers of different polymers such as NAFION™ and correlating changes of potential based on oxygen changes, which will indirectly correlate with the concentrations of creatinine.

In yet another example, sensing creatinine is provided by using sarcosine oxidase wired to at least one WE using one or more electrically coupled mediators. In this approach, concentration of creatinine will indirectly correlate with the electron transfer generated signal collected from the WE.

For the aforementioned creatinine sensor configurations based on hydrogen peroxide and/or oxygen measurements the one or more enzymes can be in a single enzyme domain, or the one or more enzymes, independently, can be in one or more enzyme domains, or any other combination thereof, in which in each layer at least one enzyme is present. For the aforementioned creatinine sensor configurations based on use of an electrically coupled sarcosine oxidase containing layer, the layer positioned adjacent to the electrode and is electrically coupled to at least a portion of the electrode surface using mediators.

In another example, the aforementioned creatinine sensor configurations can be sensed using potentiometry by using urease enzyme (UR) that creates ammonium from urea, the urea created by CRH from creatine, the creatine being formed from the interaction of creatinine with CNH. Thus, ammonium can be measured by the above configuration and correlated with the creatinine concentration. Alternatively, creatine amidohydrolase (CI) or creatinine deiminase can be used to create ammonia gas, which under physiological conditions of a transcutaneous sensor, would provide ammonium ion for signal transduction.

In yet another example, sensing creatinine is provided by using one or more enzymes and one or more cofactors. Some non-limiting examples of such configurations include creatinine deaminase (CD) providing ammonium from creatinine, glutamate dehydrogenase (GLDH) providing peroxide from the ammonium, where hydrogen peroxide correlates with levels of present creatinine. The above configuration can further include a third enzyme glutamate oxidase (GLOD) to further break down glutamate formed from the GDLH and create additional hydrogen peroxide. Such combinations of enzymes, independently, can be in one or more enzyme domains, or any other combination thereof, in which in each domain or layer, at least one enzyme is present.

In yet another example, sensing creatinine is provided by the combination of creatinine amidohydrolase (CNH), creatine kinase (CK) and pyruvate kinase (PK), where pyruvate, created by PK can be detected by one or more of either lactate dehydrogenase (LDH) or pyruvate oxidase (PDX) enzymes configured independently, where one or more of the aforementioned enzyme are present in one layer, or, in which in each of a plurality of layers comprises at least one enzyme, any other combination thereof.

In such sensor configurations where one or more cofactors and/or regenerating enzymes for the cofactors are used, providing excess amounts of one or more of NADH, NAD(P)H and ATP in any of the one or more configurations can be employed, and one or more diffusion resistance domains can be introduced to limit or prevent flux of the cofactors from their respective membrane(s). Other configurations can be used in the aforementioned configurations, such as electrode, resistance, bio-interfacing, and drug releasing membranes.

In yet another example, creatinine detection is provided by using creatinine deiminase in one or more enzyme domains and providing ammonium to the enzyme domain(s) via catalysis of creatinine. Ammonium ion can then be detected potentiometrically or by using composite electrodes that undergo redox when exposed to ammonium ion, for example NAFION™/polyaniline composite electrodes, in which polyaniline undergoes redox in the presence of ammonium at the electrode under potential. Ammonium concentration can then be correlated to creatinine concentration.

FIG. 11 depicts an exemplary continuous sensor configuration for creatinine. In the example of FIG. 11, the sensor includes a first enzyme domain 864 comprising CNH, CRH, and SOX are adjacent a working electrode WE, e.g., platinum. A second enzyme domain 865 is positioned adjacent the first enzyme domain and is more distal from the WE. One or more resistance domains (RL) 852 can be positioned adjacent the second enzyme domain or between the first and second layers. Creatinine is diffusible through the RL and the second enzyme domain to the first enzyme domain where it is converted to peroxide and transduces a signal corresponding to its concentration. Creatine is diffusible through the RL and is converted in the second enzyme domain to sarcosine and urea, the sarcosine being consumed by the sarcosine oxidase and the peroxide generated is consumed by the catalase, thus preventing transduction of the creatine signal.

For example, variations of the above configuration are possible for continuous monitoring of creatinine alone or in combination with one or more other analytes. Thus, one alternative approach to sensing creatinine could be sensing oxygen level changes electrochemically, for example in a Clark-type electrode setup. In one example, the WE can be coated with layers of different polymers, such as NAFION™ and based on changes of potential oxygen changes, the concentrations of creatinine can be correlated. In yet another example, one or more enzyme most proximal to the WE, i.e., sarcosine oxidase, can be “wired” to the electrode using one or more mediators. Each of the different enzymes in the above configurations can be distributed inside a polymer matrix or domain to provide one enzyme domain. In another example, one or more of the different enzymes discussed herein can be formed as the enzyme domain and can be formed layer by layer, in which each layer has at least one enzyme present. In an example of a “wired” enzyme configuration with a multilayered membrane, the wired enzyme domain would be most proximal to the electrode. One or more interferent layers can be deposited among the multilayer enzyme configuration so as to block of non-targeted analytes from reaching electrodes.

In one example, the aforementioned continuous creatinine sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations to provide a continuous multi-analyte sensor device as further described below. This continuous multi-analyte sensor device can further include continuous glucose monitoring capability.

### Lactose Sensor Configurations

In one example, a continuous lactose sensor configuration, alone or in combination with another analyte sensing configuration comprising one or more enzymes and/or cofactors is provided. In a general sense, a lactose sensing configuration using at least one enzyme domain comprising lactase enzyme is used for producing glucose and galactose from the lactose. The produced glucose or galactose is then enzymatically converted to a peroxide for signal transduction at an electrode. Thus, in one example, at least one enzyme domain EZL1 comprising lactase is positioned proximal to at least a portion of a WE surface capable of electrolysis of hydrogen peroxide. In one example, glucose oxidase enzyme (GOX) is included in EZL1, with one or more cofactors or electrically coupled mediators. In another example, galactose oxidase enzyme (GalOx) is included in EZL1, optionally with one or more cofactors or mediators. In one example, glucose oxidase enzyme and galactose oxidase are both included in EZL1. In one example, glucose oxidase enzyme and galactose oxidase are both included in EZL1, optionally with one or more cofactors or electrically coupled mediators.

One or more additional EZL's (e.g. EZL2) can be positioned adjacent the EZL1, where at least a portion of EZL2 is more distal from at least a portion of WE than EZL1. In one example, one or more layers can be positioned in between EZL1 and EZL2, such layers can comprise enzyme, cofactor or mediator or be essentially devoid of one or more of enzymes, cofactors or mediators. In one example, the one or more layers positioned in between EZL1 and EZL2 is essentially devoid of enzyme, e.g., no purposefully added enzyme. In one example one or layers can be positioned adjacent EZL2, being more distal from at least a portion of EZL1 than EZL2, and comprise one or more of the enzymes present in either EZL1 or EZL2.

In one example of the aforementioned lactose sensor configurations, the peroxide generating enzyme can be electrically coupled to the electrode using coupling mediators. The transduced peroxide signals from the aforementioned lactose sensor configurations can be correlated with the level of lactose present.

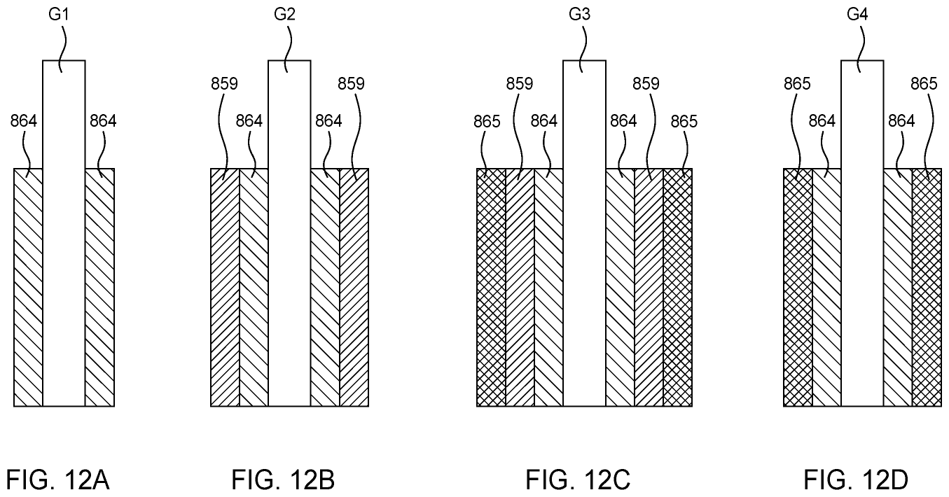

FIG. 12A-12D depict alternative continuous lactose sensor configurations. Thus, in an enzyme domain EZL1 864 most proximal to WE (G1), comprising GalOx and lactase, provides a lactose sensor that is sensitive to galactose and lactose concentration changes and is essentially non-transducing of glucose concentration. As shown in FIGS. 12B-12D, additional layers, including non-enzyme containing layers 859, and a lactase enzyme containing layer 865, and optionally, electrode, resistance, bio-interfacing, and drug releasing membranes. (not shown) are used. Since changes in physiological galactose concentration are minimal, the transduced signal would essentially be from physiological lactose fluctuations.

In one example, the aforementioned continuous lactose sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations, creatinine sensor configurations to provide a continuous multi-analyte sensor device as further described below. This continuous multi-analyte sensor device can further include continuous glucose monitoring capability. Other membranes can be used in the aforementioned sensor configuration, such as electrode, resistance, bio-interfacing, and drug releasing membranes.

### Urea Sensor Configurations

Similar approach as described above can also be used to create a continuous urea sensor. For example urease (UR), which can break down the urea and to provide ammonium can be used in an enzyme domain configuration. Ammonium can be detected with potentiometry or by using a composite electrodes, e.g., electrodes that undergo redox when exposed to ammonium. Example electrodes for ammonium signal transduction include, but are not limited to, NAFION™/polyaniline composite electrodes, in which polyaniline undergoes redox in the presence of ammonium at an applied potential, with essentially direct correlation of signal to the level of ammonium present in the surrounding. This method can also be used to measure other analytes such as glutamate using the enzyme glutaminase (GLUS).

In one example, the aforementioned continuous uric acid sensor configurations can be combined with any one of the aforementioned continuous alcohol sensor configurations and/or continuous uric acid sensor configurations and/or continuous cholesterol sensor configurations and/or continuous bilirubin/ascorbic acid sensor configurations and/or continuous ketone sensor configurations and/or continuous choline sensor configurations and/or continuous glycerol sensor configurations and/or continuous creatinine sensor configurations and/or continuous lactose sensor configurations to provide a continuous multi-analyte sensor device as further described below. This continuous multi-analyte sensor device can further include continuous glucose monitoring capability. Other membranes can be used in the aforementioned uric acid sensor configuration, such as electrode, resistance, bio-interfacing, and drug releasing membranes.

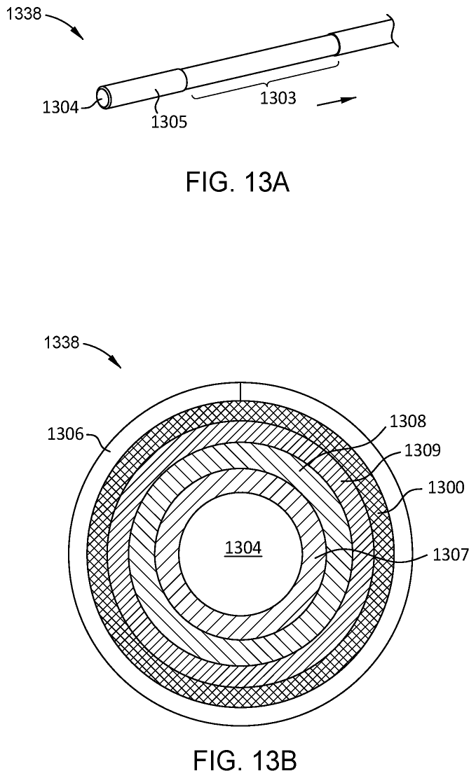

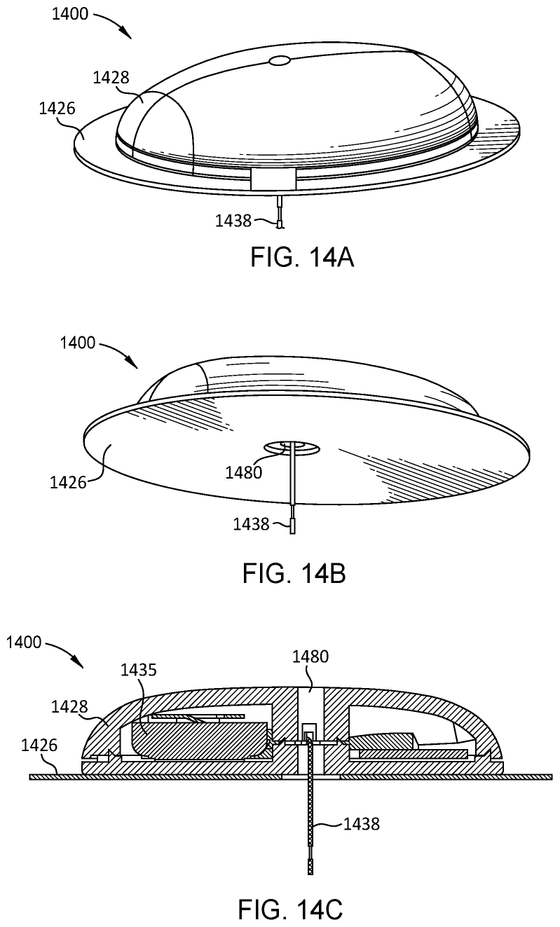

In certain embodiments, continuous analyte monitoring system 104 may be a lactate sensor, as discussed in reference to FIG. 1. FIGS. 13A-14C describe an example lactate sensor systems used to measure lactate, according to certain embodiments of the present disclosure.

FIG. 13A shows one exemplary embodiment of the physical structure of lactate sensor 1338. In this embodiment, a radial window 1303 is formed through an insulating layer 1305 to expose an electroactive working electrode of conductor material 1304. Although FIG. 13A shows a coaxial design, any form factor or shape such as a planar sheet may alternatively be used. A variety of lactate sensor designs are described in Rathee et al. “Biosensors based on electrochemical lactate detection: A comprehensive review,” Biochemistry and Biophysics Reports 5 (2016) pages 35-54, and also Rasaei et al. “Lactate Biosensors: current status and outlook” in Analytical and Bioanalytical Chemistry, September 2013, both of which are incorporated herein by reference in their entireties.

FIG. 13B is a cross-sectional view of the electroactive section of the example sensor of FIG. 13A showing the exposed electroactive surface of the working electrode surrounded by a sensing membrane in one embodiment. Such sensing membranes are present in a variety of lactate sensor designs. As shown in FIG. 13B, a sensing membrane may be deposited over at least a portion of the electroactive surfaces of the sensor (working electrode and optionally reference electrode) and provides protection of the exposed electrode surface from the biological environment, diffusion resistance of the analyte, a catalyst for enabling an enzymatic reaction, limitation or blocking of interferants, and/or hydrophilicity at the electrochemically reactive surfaces of the sensor interface.

Thus, the sensing membrane may include a plurality of domains, for example, an electrode domain 1307, an interference domain 1308, an enzyme domain 1309 (for example, including lactate oxidase), and a resistance domain 1300, and can include a high oxygen solubility domain, and/or a bioprotective domain (not shown). The membrane system can be deposited on the exposed electroactive surfaces using known thin film techniques (for example, spraying, electro-depositing, dipping, or the like). In one embodiment, one or more domains are deposited by dipping the sensor into a solution and drawing out the sensor at a speed that provides the appropriate domain thickness. However, the sensing membrane can be disposed over (or deposited on) the electroactive surfaces using any known method as will be appreciated by one skilled in the art.

The sensing membrane generally includes an enzyme domain 1309 disposed more distally situated from the electroactive surfaces than the interference domain 1308 or electrode domain 1307. In some embodiments, the enzyme domain is directly deposited onto the electroactive surfaces. In the preferred embodiments, the enzyme domain 1309 provides an enzyme such as lactose oxidase to catalyze the reaction of the analyte and its co-reactant.

The sensing membrane can also include a resistance domain 1300 disposed more distal from the electroactive surfaces than the enzyme domain 1309 because there exists a molar excess of lactate relative to the amount of oxygen in blood. However, an enzyme-based sensor employing oxygen as co-reactant is preferably supplied with oxygen in non-rate-limiting excess for the sensor to respond accurately to changes in analyte concentration rather than having the reaction unable to utilize the analyte present due to a lack of the oxygen co-reactant. This has been found to be an issue with glucose concentration monitors and is the reason why the resistance domain is included. Specifically, when a glucose-monitoring reaction is oxygen limited, linearity is not achieved above minimal concentrations of glucose. Without a semipermeable membrane situated over the enzyme domain to control the flux of glucose and oxygen, a linear response to glucose levels can be obtained only for glucose concentrations of up to about 2 or 3 mM. However, in a clinical setting, a linear response to glucose levels is desirable up to at least about 20 mM. To allow accurate determination of higher glucose levels, the resistance domain in the glucose monitoring context can be 200 times more permeable to oxygen than glucose. This allows an oxygen concentration high enough to make the glucose concentration the determining factor in the rate of the detected electrochemical reaction.

In some embodiments, for the lactate sensors described herein, the resistance domain can be thinner, and have a smaller difference in analyte vs. oxygen permeability, such as 50:1, or 10:1 oxygen to lactate permeability. In some embodiments, this makes the lactate sensor more sensitive to low lactate levels such as 0.5 mM or lower up to 3 or 4 mM. The resistance domain may be configured such that lactate is the rate limiting reactant at 3 mM lactate or lower, thus allowing accurate threshold detection at around 2 mM. The resistance domain may further be configured to allow oxygen to be the rate limiting reactant at lactate concentrations greater than 10 mM. These ranges may be narrowed further in some embodiments, for example the resistance domain may be configured such that lactate is the rate limiting reactant at 4 mM lactate or lower, and such that oxygen is the rate limiting reactant at lactate concentrations greater than 6 mM. In this way, the sensor itself can be optimized for early sepsis detection. It will also be appreciated that in addition to lactate, other analyte sensors can be combined with the lactate sensor described herein, such as sensors suitable for ketones, ethanol, glycerol, glucose, hormones, viruses, or any other biological component of interest.

FIGS. 14A-14C illustrate an exemplary implementation of a sensor system 104 implemented as a wearable device such as an on-skin sensor assembly 1400. As shown in FIGS. 14A-14B, on-skin sensor assembly comprises a housing 1428. An adhesive patch 1426 can couple the housing 1428 to the skin of the host. The adhesive 1426 can be a pressure sensitive adhesive (e.g., acrylic, rubber based, or other suitable type) bonded to a carrier substrate (e.g., spun lace polyester, polyurethane film, or other suitable type) for skin attachment. The housing 1428 may include a through-hole 1480 that cooperates with a sensor inserter device (not shown) that is used for implanting the sensor 1338 under the skin of a subject.

The wearable sensor assembly 1400 includes sensor electronics 1435 operable to measure and/or analyze lactate concentration indicators sensed by lactate sensor 1438. As shown in FIG. 14C, in this implementation the sensor 1338 extends from its distal end up into the through-hole 1480 and is routed to a sensor electronics 1435, typically mounted on a printed circuit board 1435 inside the enclosure 1428. The sensor electrodes are connected to the sensor electronics 1435. These kinds of analyte monitors are currently used in commercially available glucose monitoring systems used by diabetics, and the design principles used there can be used for an lactate monitor as well.

The housing 1428 of the sensor assembly 1400 can include a user interface for delivering messages to the patient regarding sepsis status. Because the lactate sensors described herein may, in some examples, not be a monitor that a patient will wear regularly as is the case with glucose monitors, in such examples, they may not need to include many of the features present in other monitor types such as regular wireless transmission of analyte concentration data. Accordingly, a simple user interface to just deliver warnings can be implemented. In some embodiments, the user interface could be a single light-emitting diode (LED) that is illuminated when the sensor electronics determines sepsis risk is present. Two LEDs or a two-color LED could be green when the monitor is operational and detects low risk, and red when a sepsis risk is detected and a warning is issued. The monitor may be configured to revert back to a green or low risk condition if measurements return to values appropriate for that output. To provide additional flexibility in delivering messages to patients such as error messages, time remaining to wear the device, etc., a simple dot matrix character display could be used (for example less than 200 pixels a side or a configurable 20 character LCD) that would still be inexpensive and power efficient.

### Additional Considerations

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ containing,′ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A monitoring system, comprising:
a continuous analyte sensor configured generate analyte measurements associated with analyte levels of a patient; and
a sensor electronics module coupled to the continuous analyte sensor and configured to receive and process the analyte measurements.

2. The monitoring system of claim 1, wherein the continuous analyte sensor comprises:
an electroactive working electrode of conductor material configured to be inserted into a skin of the patient, wherein the electroactive working electrode is surrounded by a sensing membrane for sensing the analyte levels.

3. The monitoring system of claim 1, wherein:
the continuous analyte sensor is a continuous lactate sensor, and
the analyte measurements include lactate measurements.

4. The monitoring system of claim 3, further comprising:
a memory comprising executable instructions;
one or more processors in data communication with the sensor electronics module and configured by the executable instructions to:
receive analyte data from the sensor electronics module, the analyte data comprising the lactate measurements associated with at least a first time period;
process the analyte data from the at least the first time period to determine at least one lactate derived metric; and
generate a disease prediction using the at least one lactate derived metric.

5. The monitoring system of claim 4, wherein the processor is further configured to generate one or more recommendations for treatment based, at least in part, on the disease prediction.

6. The monitoring system of claim 5, wherein the one or more recommendations for treatment comprise at least one of:
lifestyle modification recommendations;
drug prescription recommendations;
surgical procedure recommendations; or
medical device recommendations for use by the patient.

7. The monitoring system of claim 4, wherein the disease prediction comprises at least one of:
an indication of a presence of liver disease in the patient;
an indication of a severity of the liver disease in the patient;
a score associated with the liver disease of the patient;
an indication of a level of risk of the patient being diagnosed with the liver disease;
an indication of a level of improvement or deterioration of the liver disease in the patient;
an indication of a level of improvement or deterioration of the liver disease in the patient in response to an investigational drug and/or device intervention, wherein the device intervention comprises invention by a gastric bypass device, an electro-muscular stimulation device, or a TENS device;
a mortality risk of the patient; or
an identification of one or more diseases associated with the liver disease of the patient and an associated risk of the patient being diagnosed with the one or more diseases.

8. The monitoring system of claim 7, wherein the indication of the level of improvement or the deterioration of the liver disease in the patient is based, at least in part, on at least one of:
a procedure previously performed on the patient;
a drug previously ingested by the patient.

9. The monitoring system of claim 4, wherein the at least one lactate derived metric comprises at least one of a lactate clearance rate, a lactate area under a curve, a lactate baseline, a lactate rate of change, or a postprandial lactate level.

10. The monitoring system of claim 4, further comprising:
one or more non-analyte sensors configured to generate non-analyte sensor data during the first time period.

11. The monitoring system of claim 10, wherein:
the at least one lactate derived metric comprises at least a first lactate clearance rate; and
the processor being configured to process the analyte data from the at least the first time period to determine the at least one lactate clearance rate comprises the processor being configured to:
identify at least one period of increased lactate of the patient during the at least the first time period;
calculate a first lactate clearance rate of the patient after the at least one period of increased lactate; and
correct the first lactate clearance rate of the patient to isolate lactate clearance by a liver of the patient based, at least in part, on the non-analyte sensor data.

12. The monitoring system of claim 11, wherein the at least one period of increased lactate is due to at least one of:
physical exertion by the patient; or
consumption of lactate by the patient.

13. The monitoring system of claim 11, wherein the processor being configured to calculate the first lactate clearance rate of the patient after the at least one period of increased lactate comprises the processor being configured to:
determine a maximum lactate level of the patient during the at least one period of increased lactate;
determine an amount of time the maximum lactate level takes to decrease to a percentage of a baseline lactate level or a percentage of the maximum lactate level of the patient after the at least one period of increased lactate; and
calculate the first lactate clearance rate of the patient using the determined maximum lactate level of the patient, the baseline lactate level of the patient, and the determined amount of time the maximum lactate level takes to decrease to the percentage of the baseline lactate level of the patient.

14. The monitoring system of claim 11, wherein the processor being configured to correct the first lactate clearance rate of the patient comprises the processor being configured to:
identify the at least one period of increased lactate is due to physical exertion by the patient using the non-analyte sensor data;
compare the non-analyte sensor data with other non-analyte sensor data for one or more other periods of increased lactate due to physical exertion and having pre-determined lactate clearance rate breakdowns, wherein the pre-determined lactate clearance rate breakdowns represent a breakdown of lactate clearance by at least one of the liver, kidneys, muscles, and a heart of the patient; and
determine a second lactate clearance rate indicative of lactate clearance by only the liver of the patient based, at least in part, on the comparison, and
wherein the disease prediction is generated using at least the analyte data for the one or more analytes and the second lactate clearance rate.

15. The monitoring system of claim 11, wherein the processor being configured to correct the first lactate clearance rate of the patient comprises the processor being configured to:
identify the at least one period of increased lactate is not due to physical exertion by the patient, using the non-analyte sensor data;
compare the data generated by the non-analyte sensor data with other non-analyte sensor data for one or more other periods of increased lactate not due to physical exertion and having pre-determined lactate clearance rate breakdowns, wherein the pre-determined lactate clearance rate breakdowns represent a breakdown of lactate clearance by at least one of the liver, kidneys, muscles, and a heart of the patient;
determine a second lactate clearance rate indicative of lactate clearance by only the liver of the patient based, at least in part, on the comparison; and
wherein the disease prediction is generated using at least the analyte data for one or more analytes and the second lactate clearance rate.

16. The monitoring system of claim 4, wherein the disease prediction is generated using a model trained using training data, wherein the training data comprises records of historical patients with varying stages of liver disease.

17. The monitoring system of claim 4, wherein the processor is further configured to:
obtain at least one of demographic information, food consumption information, activity level information, or medication information related to the patient, and
wherein the disease prediction is generated further using at least one of the demographic information, the food consumption information, the activity level information, or the medication information.

18. The monitoring system of claim 4, wherein the one or more analytes further include at least one of glucose or ketones.

19. The monitoring system of claim 4, wherein the one or more analytes of the patient are monitored continuously, semi-continuously, or periodically.
